SEQUENCE LISTING (31 sequences)

SEQ ID NO: 1 - DNA, 44 bp, synthetic construct
  misc_feature 1..44: note = Description of Artificial Sequence: Synthetic primer
  source 1..44: mol_type = other DNA
atggtgagcaagggcgaggagctgttcaccggggtggtgcccat

SEQ ID NO: 2 - DNA, 44 bp, synthetic construct
  misc_feature 1..44: note = Description of Artificial Sequence: Synthetic primer
  source 1..44: mol_type = other DNA
atgtttagcaagggcgaggagctgttcaccggggtggtgcccat

SEQ ID NO: 3 - DNA, 44 bp, synthetic construct
  misc_feature 1..44: note = Description of Artificial Sequence: Synthetic primer
  source 1..44: mol_type = other DNA
atgttaagcaagggcgaggagctgttcaccggggtggtgcccat

SEQ ID NO: 4 - DNA, 44 bp, synthetic construct
  misc_feature 1..44: note = Description of Artificial Sequence: Synthetic primer
  source 1..44: mol_type = other DNA
atgattagcaagggcgaggagctgttcaccggggtggtgcccat

SEQ ID NO: 5 - DNA, 44 bp, synthetic construct
  misc_feature 1..44: note = Description of Artificial Sequence: Synthetic primer
  source 1..44: mol_type = other DNA
atgtctagcaagggcgaggagctgttcaccggggtggtgcccat

SEQ ID NO: 6 - DNA, 44 bp, synthetic construct
  misc_feature 1..44: note = Description of Artificial Sequence: Synthetic primer
  source 1..44: mol_type = other DNA
atgcctagcaagggcgaggagctgttcaccggggtggtgcccat

SEQ ID NO: 7 - DNA, 44 bp, synthetic construct
  misc_feature 1..44: note = Description of Artificial Sequence: Synthetic primer
  source 1..44: mol_type = other DNA
atgactagcaagggcgaggagctgttcaccggggtggtgcccat

SEQ ID NO: 8 - DNA, 44 bp, synthetic construct
  misc_feature 1..44: note = Description of Artificial Sequence: Synthetic primer
  source 1..44: mol_type = other DNA
atggctagcaagggcgaggagctgttcaccggggtggtgcccat

SEQ ID NO: 9 - DNA, 44 bp, synthetic construct
  misc_feature 1..44: note = Description of Artificial Sequence: Synthetic primer
  source 1..44: mol_type = other DNA
atgtatagcaagggcgaggagctgttcaccggggtggtgcccat

SEQ ID NO: 10 - DNA, 44 bp, synthetic construct
  misc_feature 1..44: note = Description of Artificial Sequence: Synthetic primer
  source 1..44: mol_type = other DNA
atgcatagcaagggcgaggagctgttcaccggggtggtgcccat

SEQ ID NO: 11 - DNA, 44 bp, synthetic construct
  misc_feature 1..44: note = Description of Artificial Sequence: Synthetic primer
  source 1..44: mol_type = other DNA
atgcaaagcaagggcgaggagctgttcaccggggtggtgcccat

SEQ ID NO: 12 - DNA, 44 bp, synthetic construct
  misc_feature 1..44: note = Description of Artificial Sequence: Synthetic primer
  source 1..44: mol_type = other DNA
atgaatagcaagggcgaggagctgttcaccggggtggtgcccat

SEQ ID NO: 13 - DNA, 44 bp, synthetic construct
  misc_feature 1..44: note = Description of Artificial Sequence: Synthetic primer
  source 1..44: mol_type = other DNA
atgaaaagcaagggcgaggagctgttcaccggggtggtgcccat

SEQ ID NO: 14 - DNA, 44 bp, synthetic construct
  misc_feature 1..44: note = Description of Artificial Sequence: Synthetic primer
  source 1..44: mol_type = other DNA
atggatagcaagggcgaggagctgttcaccggggtggtgcccat

SEQ ID NO: 15 - DNA, 44 bp, synthetic construct
  misc_feature 1..44: note = Description of Artificial Sequence: Synthetic primer
  source 1..44: mol_type = other DNA
atggaaagcaagggcgaggagctgttcaccggggtggtgcccat

SEQ ID NO: 16 - DNA, 44 bp, synthetic construct
  misc_feature 1..44: note = Description of Artificial Sequence: Synthetic primer
  source 1..44: mol_type = other DNA
atgtgtagcaagggcgaggagctgttcaccggggtggtgcccat

SEQ ID NO: 17 - DNA, 44 bp, synthetic construct
  misc_feature 1..44: note = Description of Artificial Sequence: Synthetic primer
  source 1..44: mol_type = other DNA
atgtggagcaagggcgaggagctgttcaccggggtggtgcccat

SEQ ID NO: 18 - DNA, 44 bp, synthetic construct
  misc_feature 1..44: note = Description of Artificial Sequence: Synthetic primer
  source 1..44: mol_type = other DNA
atgcgtagcaagggcgaggagctgttcaccggggtggtgcccat

SEQ ID NO: 19 - DNA, 44 bp, synthetic construct
  misc_feature 1..44: note = Description of Artificial Sequence: Synthetic primer
  source 1..44: mol_type = other DNA
atgggtagcaagggcgaggagctgttcaccggggtggtgcccat

SEQ ID NO: 20 - DNA, 62 bp, synthetic construct
  misc_feature 1..62: note = Description of Artificial Sequence: Synthetic oligonucleotide
  source 1..62: mol_type = other DNA
agacaatcaaccatttggggtggacagccttgacctctagacttcggcattttttttttttt

SEQ ID NO: 21 - DNA, 112 bp, synthetic construct
  misc_feature 1..112: note = Description of Artificial Sequence: Synthetic oligonucleotide
  source 1..112: mol_type = other DNA
cgggatccttatcgtcatcgtcgtacagatcccgacccatttgctgtccaccagtcatgctagccataccatgatgatgatgatgatgagaaccccgcattttttttttttt

SEQ ID NO: 22 - DNA, 19 bp, synthetic construct
  misc_feature 1..19: note = Description of Artificial Sequence: Synthetic primer
  source 1..19: mol_type = other DNA
atgcggggttctcatcatc

SEQ ID NO: 23 - DNA, 20 bp, synthetic construct
  misc_feature 1..20: note = Description of Artificial Sequence: Synthetic primer
  source 1..20: mol_type = other DNA
cgggatccttatcgtcatcg

SEQ ID NO: 24 - protein, 7 aa, synthetic construct
  REGION 1..7: note = Description of Artificial Sequence: Synthetic peptide
AWIKREQ

SEQ ID NO: 25 - protein, 7 aa, synthetic construct
  REGION 1..7: note = Description of Artificial Sequence: Synthetic peptide
  VARIANT 1: note = any amino acid
XWIKREQ

SEQ ID NO: 26 - protein, 7 aa, synthetic construct
  REGION 1..7: note = Description of Artificial Sequence: Synthetic peptide
  VARIANT 2: note = any amino acid
AXIKREQ

SEQ ID NO: 27 - protein, 7 aa, synthetic construct
  REGION 1..7: note = Description of Artificial Sequence: Synthetic peptide
  VARIANT 3: note = any amino acid
AWXKREQ

SEQ ID NO: 28 - protein, 7 aa, synthetic construct
  REGION 1..7: note = Description of Artificial Sequence: Synthetic peptide
  VARIANT 4: note = any amino acid
AWIXREQ

SEQ ID NO: 29 - protein, 7 aa, synthetic construct
  REGION 1..7: note = Description of Artificial Sequence: Synthetic peptide
  VARIANT 5: note = any amino acid
AWIKXEQ

SEQ ID NO: 30 - protein, 7 aa, synthetic construct
  REGION 1..7: note = Description of Artificial Sequence: Synthetic peptide
  VARIANT 6: note = any amino acid
AWIKRXQ

SEQ ID NO: 31 - protein, 7 aa, synthetic construct
  REGION 1..7: note = Description of Artificial Sequence: Synthetic peptide
  VARIANT 7: note = any amino acid
AWIKREX

(19) United States
(12) Patent Application Publication
(10) Pub. No.: US 2024/0158955 A1
(43) Pub. Date: May 16, 2024

(54) OLIGONUCLEIC ACID VARIANT LIBRARIES AND SYNTHESIS THEREOF

(71) Applicant: TWIST BIOSCIENCE CORPORATION, South San Francisco, CA (US)

(72) Inventors: Anthony COX, Mountain View, CA (US); Sebastian TREUSCH, San Francisco, CA (US); Siyuan CHEN, San Mateo, CA (US)

(21) Appl. No.: 18/356,787

(22) Filed: Jul. 21, 2023

Related U.S. Application Data
(62) Division of application No. 17/068,551, filed on Oct. 12, 2020, now Pat. No. 11,807,956.
Application No. 17/068,551 is a continuation of application No. 15/268,422, filed on Sep. 16, 2016, now Pat. No. 10,844,373.
(60) Provisional application No. 62/354,034, filed on Jun. 23, 2016.
(60) Provisional application No. 62/263,548, filed on Dec. 4, 2015.
(60) Provisional application No. 62/220,879, filed on Sep. 18, 2015.

(51) Int. Cl. C40B 50/06; C12N 15/10; C12N 15/113; C12Q 1/6876; C40B 50/08; C40B 50/14; G01N 33/50

(52) U.S. Cl. CPC C40B 50/06; C12N 15/1079; C12N 15/1093; C12N 15/113; C12Q 1/6876; C40B 50/08; C40B 50/14; G01N 33/502; G01N 33/5029; G01N 33/5041; C12N 2320/30; C12N 2330/31; C12Q 1/6886

(57) ABSTRACT

Disclosed herein are methods for the generation of highly accurate oligonucleic acid libraries encoding for predetermined variants of a nucleic acid sequence. The degree of variation may be complete, resulting in a saturated variant library, or less than complete, resulting in a selective library of variants. The variant oligonucleic acid libraries described herein may designed for further processing by transcription or translation. The variant oligonucleic acid libraries described herein may be designed to generate variant RNA, DNA and/or protein populations. Further provided herein are method for identifying variant species with increased or decreased activities, with applications in regulating biological functions and the design of therapeutics for treatment or reduction of disease.

### CROSS-REFERENCE

This application is a divisional of U.S. patent application Ser. No. 17/068,551, filed Oct. 12, 2020, which is a continuation application of U.S. patent application Ser. No. 15/268,422, filed Sep. 16, 2016, now U.S. Pat. No. 10,844,373 issued Nov. 24, 2020, which claims the benefit of U.S. Provisional Application No. 62/220,879, filed Sep. 18, 2015, U.S. Provisional Application No. 62/263,548, filed Dec. 4, 2015, and U.S. Provisional Application No. 62/354,034, filed Jun. 23, 2016, all of which are incorporated herein by reference.

### SEQUENCE LISTING

The instant application contains a Sequence Listing which has been submitted electronically in XML format and is hereby incorporated by reference in its entirety. Said XML copy, created on Feb. 2, 2024, is named Sequence_Listing.xml, and is 44,232 bytes in size.

### BACKGROUND

The cornerstone of synthetic biology is the design, build, and test process—an iterative process that requires DNA, to be made accessible for rapid and affordable generation and optimization of these custom pathways and organisms. In the design phase, the A, C, T and G nucleotides that constitute DNA are formulated into the various gene sequences that would comprise the locus or the pathway of interest, with each sequence variant representing a specific hypothesis that will be tested. These variant gene sequences represent subsets of sequence space, a concept that originated in evolutionary biology and pertains to the totality of sequences that make up genes, genomes, transcriptome and proteome.

Many different variants are typically designed for each design-build-test cycle to enable adequate sampling of sequence space and maximize the probability of an optimized design. Though straightforward in concept, process bottlenecks around speed, throughput and quality of conventional synthesis methods dampen the pace at which this cycle advances, extending development time. The inability to sufficiently explore sequence space due to the high cost of acutely accurate DNA and the limited throughput of current synthesis technologies remains the rate-limiting step.

Beginning with the build phase, two processes are noteworthy: oligonucleic acid synthesis and gene synthesis. Historically, synthesis of different gene variants was accomplished through molecular cloning. While robust, this approach is not scalable. Early chemical gene synthesis efforts focused an producing a large number of oligonucleic acids with overlapping sequence homology. These were then pooled and subjected to multiple rounds of polymerase chain reaction (PCR), enabling concatenation of the overlapping oligonucleic acids into a full length double stranded gene. A number of factors hinder this method, including time-consuming and labor-intensive construction, requirement of high volumes of phosphoramidites, an expensive raw material, and production of nanomole amounts of the final product, significantly less than required for downstream steps, and a large number of separate oligonucleic acids required one 96 well plate to set up the synthesis of one gene.

Synthesizing oligonucleic acids on microarrays provided a significant increase in the throughput of gene synthesis. A large number of oligonucleic acids could be synthesized on the microarray surface, then cleaved off and pooled together. Each oligonucleic acid destined for a specific gene contains a unique barcode sequence that enabled that specific subpopulation of oligonucleotides to be depooled and assembled into the gene of interest. In this phase of the process, each subpool is transferred into one well in a 96 well plate, increasing throughput to 96 genes. While this is two orders of magnitude higher in throughput than the classical method, it still does not adequately support the design, build, test cycles that require thousands of sequences at one time due to a lack of cost efficiency and slow turnaround times.

### BRIEF SUMMARY

Provided herein are methods for modulating protein activity, comprising: providing predetermined sequences encoding for at least about 30,000 non-identical oligonucleic acids, wherein each of the at least about 30,000 non-identical oligonucleic encodes for a variant codon sequence compared to a single reference sequence; providing a structure having a surface; synthesizing the at least about 30,000 non-identical oligonucleic acids, wherein each of the at least about 30,000 non-identical oligonucleic acids extends from the surface; mixing the least about 30,000 non-identical oligonucleic acids with a DNA polymerase and the single reference sequence to form a library of variant nucleic acids; transferring the library of variant nucleic acids to cells and expressing a plurality of variant proteins; and identifying an activity associated with a variant protein of the plurality of variant proteins, wherein the activity is modulated relative to a protein encoded by the single reference sequence. Further provided herein are methods wherein the activity comprises cellular reproduction, growth, adhesion, death, migration, energy production, oxygen utilization, metabolic activity, cell signaling, aging, response to free radical damage, or any combination thereof. Further provided herein are methods wherein the cells are eukaryotic cells or prokaryotic cells. Further provided herein are methods wherein the cells are bacterial, plant, mouse, or primate cells. Further provided herein are methods wherein the library of variant nucleic acids encodes sequences for variant genes or fragments thereof. Further provided herein are methods wherein the variant protein is an antibody, enzyme or peptide. Further provided herein are methods wherein the variant protein has enhanced or reduced binding affinity for another molecule. Further provided herein are methods wherein the variant protein has enhanced or reduced enzymatic activity. Further provided herein are methods wherein single variant nucleic acid encoded by the library of variant nucleic acids is administered to a subject in need thereof. Further provided herein are methods wherein the at least about 30,000 non-identical oligonucleic acids have an aggregate error rate of less than 1 in 1000 bases compared to predetermined sequences for the plurality of non-identical oligonucleic acids.

Provided herein are methods for modulating cellular activity, comprising: providing predetermined sequences encoding for at least about 30,000 non-identical oligonucleic acids, wherein each of the at least about 30,000 non-identical oligonucleic encodes for a predetermined variant sequence compared to a single reference sequence; providing a structure having a surface; synthesizing the at least about 30,000 non-identical oligonucleic acids, wherein each of the at least about 30,000 non-identical oligonucleic acids extends from the surface, and wherein the at least about 30,000 non-identical oligonucleic acids have an aggregate error rate of less than 1 in 1000 bases compared to predetermined sequences for the plurality of non-identical oligonucleic acids; mixing the least about 30,000 non-identical oligonucleic acids with a DNA polymerase and the single reference sequence to form a library of variant nucleic acids; transferring the library of variant nucleic acids to a first set of cells; and measuring a change in a cellular activity, wherein the cellular activity is measured for the first set of cells or a second set of cells, wherein the second set of cells are treated at least one expression product isolated from the first set of cells. Further provided herein are methods wherein the cellular activity comprises reproduction, growth, adhesion, death, migration, energy production, oxygen utilization, metabolic activity, cell signaling, response to free radical damage, or any combination thereof. Further provided herein are methods wherein the first set of cells or the second set of cells are eukaryotic cells or prokaryotic cells. Further provided herein are methods wherein the first set of cells or the second set of cells are bacterial, plant, mouse, or primate cells. Further provided herein are methods wherein the library of variant nucleic acids encodes sequences for variant genes or fragments thereof. Further provided herein are methods comprising translation of each of the variant genes to form a protein. Further provided herein are methods wherein the protein is an antibody, enzyme, or peptide. Further provided herein are methods wherein the library of variant nucleic acids encodes for at least a portion of a variable region or constant region of the antibody. Further provided herein are methods wherein the library of variant nucleic acids encodes for at least one CDR region of the antibody. Further provided herein are methods wherein the protein has enhanced or reduced binding affinity for another molecule, or wherein the protein has enhanced or reduced enzymatic activity. Further provided herein are methods wherein the library of variant nucleic acids encodes for a transcription regulatory sequence. Further provided herein are methods wherein the transcription regulatory sequence is a promoter, UTR, or a terminator sequence. Further provided herein are methods wherein the library of variant nucleic acids encodes for a sequence that, when transcribed, encodes for mRNA, miRNA, or shRNA.

Provided herein are methods for treatment or reduction of a disease state, comprising: providing predetermined sequences encoding for at least about 30,000 non-identical oligonucleic acids, wherein each of the at least about 30,000 non-identical oligonucleic encodes for a variant codon sequence compared to a single reference sequence; providing a structure having a surface; synthesizing the at least about 30,000 non-identical oligonucleic acids, wherein each of the at least about 30,000 non-identical oligonucleic acids extends from the surface; mixing the least about 30,000 non-identical oligonucleic acids with a DNA polymerase and the single reference sequence to form a library of variant nucleic acids; transferring the library of variant nucleic acids to cells obtained from a subject; identifying a reduction in harmful activity associated with a variant nucleic acid encoded by the library of variant nucleic acids; and administering the variant nucleic acid encoded by the library of variant nucleic acids to a subject in need thereof, thereby treating or reducing the disease state. Further provided herein are methods wherein the disease state is associated with a cell proliferative, autoimmune, viral, or bacterial disorder. Further provided herein are methods wherein the at least about 30,000 non-identical oligonucleic acids have an aggregate error rate of less than 1 in 1000 bases compared to predetermined sequences for the plurality of non-identical oligonucleic acids.

Provided herein are methods for nucleic acid synthesis, comprising: providing predetermined sequences encoding for a plurality of non-identical oligonucleic acids, wherein each of the non-identical oligonucleic acids is at least 20 bases in length, and wherein the plurality of non-identical oligonucleic acids encodes for about 19 variants for each of at least 3 codons for at least one sequence, and wherein the plurality of non-identical oligonucleic acids collectively encodes for at least one gene and variants thereof; providing a structure having a surface; synthesizing the plurality of non-identical oligonucleic acids, wherein each of the non-identical oligonucleic acids extends from the surface; and assembling a library of variant nucleic acids from the plurality of non-identical oligonucleic acids. Further provided herein are methods wherein the plurality of non-identical oligonucleic acids comprises at least 75,000 non-identical oligonucleic acids. Further provided herein are methods wherein a subset of the plurality of non-identical oligonucleic acids collectively encodes for a single gene and variants thereof is located within a single cluster on the surface of the structure. Further provided herein are methods wherein the surface of the structure comprises at least 6000 of the single clusters. Further provided herein are methods wherein each cluster is located within a channel about 0.5 to 2 mm in diameter. Further provided herein are methods wherein the single cluster comprises 50 to 500 loci for nucleic acid extension. Further provided herein are methods wherein the plurality of non-identical oligonucleic acids collectively encode for variants of more than one gene. Further provided herein are methods wherein the plurality of non-identical oligonucleic acids collectively encode for variants of at least 5,000 genes. Further provided herein are methods wherein the library of variant nucleic acids encodes for at least a portion of an enzyme, peptide, or antibody. Further provided herein are methods wherein each variant nucleic acid encodes sequence for at least 85% of a gene. Further provided herein are methods wherein are protein libraries generated by expression of a plurality of expression constructs collectively comprising a non-identical oligonucleic acid described herein.

Provided herein are oligonucleic acid libraries, comprising a plurality of non-identical oligonucleic acids, wherein each of the non-identical oligonucleic acids is at least 12 bases in length, wherein the plurality of non-identical oligonucleic acids encodes for about 19 variants for each of at least 3 codons, and wherein the plurality of non-identical oligonucleic acids has an aggregate error rate of less than 1 in 1000 bases compared to predetermined sequences for the plurality of non-identical oligonucleic acids. Further provided herein are oligonucleic acid libraries, wherein the plurality of non-identical oligonucleic acids has an aggregate error rate of less than 1 in 1500 bases compared to predetermined sequences for the plurality of non-identical oligonucleic acids. Further provided herein are oligonucleic acid libraries, wherein the plurality of non-identical oligonucleic acids has an aggregate error rate of less than 1 in 2000 bases compared to predetermined sequences for the plurality of non-identical oligonucleic acids. Further provided herein are oligonucleic acid libraries, wherein each of the plurality of non-identical oligonucleic acids is at least 30 bases in length. Further provided herein are oligonucleic acid libraries, wherein each of the plurality of non-identical oligonucleic acids is at least 50 bases in length. Further provided herein are oligonucleic acid libraries, wherein each of the plurality of non-identical oligonucleic acids is 12 to 100 bases in length. Further provided herein are oligonucleic acid libraries, wherein the plurality of non-identical oligonucleic acids collectively encodes sequence for at least 85% of a gene. Further provided herein are oligonucleic acid libraries, wherein the plurality of non-identical oligonucleic acids collectively encodes sequence for a plurality of exons in a gene. Further provided herein are oligonucleic acid libraries, wherein the gene encodes for at least a portion of an antibody, enzyme, or peptide. Further provided herein are oligonucleic acid libraries, wherein the plurality of non-identical oligonucleic acids comprises oligonucleic acids that encode for a variable region or constant region of the antibody. Further provided herein are oligonucleic acid libraries, wherein the plurality of non-identical oligonucleic acids comprises oligonucleic acids that encode for at least one CDR region of the antibody. Further provided herein are oligonucleic acid libraries, wherein the plurality of non-identical oligonucleic acids collectively encodes sequence for one or more segments of an expression cassette. Further provided herein are oligonucleic acid libraries, wherein the expression cassette comprises at least one promoter region and the plurality of non-identical oligonucleic acids comprises oligonucleic acids that encode for at least a portion of the promoter region. Further provided herein are oligonucleic acid libraries, wherein the expression cassette comprises two promoter regions. Further provided herein are oligonucleic acid libraries, wherein the at least 3 codons are consecutive. Further provided herein are oligonucleic acid libraries, wherein the at least 3 codons are not consecutive. Further provided herein are oligonucleic acid libraries, wherein at least 2 of the at least 3 codons are separated by at least one codon position from each other. Further provided herein are oligonucleic acid libraries, wherein the library comprises non-identical oligonucleic acids encoding for codon variants in 4, 5, 6, 7, 8, 9 or 10 codons. Further provided herein are oligonucleic acid libraries, wherein the plurality of non-identical oligonucleic acids encodes for all possible codon variants in at least 4 codons. Further provided herein are oligonucleic acid libraries, wherein none of the non-identical oligonucleic acids encode codons for more than three histidine residues. Further provided herein are oligonucleic acid libraries, wherein none of the non-identical oligonucleic acids encode codons for more than four histidine residues.

Provided herein are oligonucleic acid libraries, comprising at least 75,000 non-identical oligonucleic acids, wherein each of the at least 75,000 non-identical oligonucleic acids is at least 30 bases in length, wherein the at least 75,000 of non-identical oligonucleic acids encode for at least 3 variants for each of at least 3 codons for at least one sequence, and wherein the at least 75,000 of non-identical oligonucleic acids have an aggregate error rate of less than 1 in 1000 bases compared to predetermined sequences for the plurality of non-identical oligonucleic acids. Further provided herein are oligonucleic acid libraries, wherein the at least 75,000 of non-identical oligonucleic acids has an aggregate error rate of less than 1 in 1500 bases compared to predetermined sequences for the at least 75,000 of non-identical oligonucleic acids. Further provided herein are oligonucleic acid libraries, wherein each of the at least 75,000 of non-identical oligonucleic acids comprises at least 50 bases in length. Further provided herein are oligonucleic acid libraries, wherein the at least 75,000 of non-identical oligonucleic acids collectively encodes sequence for at least 85% of a gene. Further provided herein are oligonucleic acid libraries, wherein the at least 75,000 of non-identical oligonucleic acids collectively encodes sequence for a plurality of exons in the same gene. Further provided herein are oligonucleic acid libraries, wherein the gene encodes for at least a portion of an antibody, enzyme, or adaptor protein. Further provided herein are oligonucleic acid libraries, wherein the at least 75,000 of non-identical oligonucleic acids comprise oligonucleic acids that encode for a variable region or constant region of the antibody. Further provided herein are oligonucleic acid libraries, wherein the at least 75,000 of non-identical oligonucleic acids comprises oligonucleic acids that encode for at least one complementarity-determining region (CDR) of the antibody. Further provided herein are oligonucleic acid libraries, wherein the at least 75,000 of non-identical oligonucleic acids collectively encodes sequence for one or more segments of an expression cassette. Further provided herein are oligonucleic acid libraries, wherein the expression cassette comprises at least one promoter region and the at least 75,000 of non-identical oligonucleic acids comprises oligonucleic acids that encode for at least a portion of the promoter region. Further provided herein are oligonucleic acid libraries, wherein the expression cassette comprises two promoter regions. Further provided herein are oligonucleic acid libraries, wherein the at least 3 codons are consecutive. Further provided herein are oligonucleic acid libraries, wherein the at least 3 codons are not consecutive. Further provided herein are oligonucleic acid libraries, wherein at least 2 of the at least 3 codons are separated by at least one codon position from each other. Further provided herein are oligonucleic acid libraries, wherein the at least 75,000 of non-identical oligonucleic acids encodes for all possible codon variants in at least 3 codons. Further provided herein are oligonucleic acid libraries, wherein none of the non-identical oligonucleic acids encode codons for more than three histidine residues. Further provided herein are oligonucleic acid libraries, wherein none of the non-identical oligonucleic acids encode codons for more than four histidine residues. Further provided herein are oligonucleic acid libraries, wherein the library comprises at least 100,000 non-identical oligonucleic acids. Further provided herein are oligonucleic acid libraries, wherein the library comprises at least 700,000 non-identical oligonucleic acids. Further provided herein are oligonucleic acid libraries, wherein the library comprises at least 1,000,000 non-identical oligonucleic acids.

Provided herein are oligonucleic acid libraries, comprising a plurality of non-identical oligonucleic acids, wherein each non-identical oligonucleic acid is about 20 to 130 bases in length, wherein the plurality of non-identical oligonucleic acids collectively encode for about 19 variants for each of at least 3 codons for at least one sequence, and wherein the plurality of non-identical oligonucleic acids has an aggregate error rate of less than 1 in 1000 bases compared to predetermined sequences for the plurality of non-identical oligonucleic acids. Further provided herein are oligonucleic acid libraries, wherein the plurality of non-identical oligonucleic acids comprises 50 to 500 non-identical oligonucleic acids. Further provided herein are oligonucleic acid libraries, wherein the plurality of non-identical oligonucleic acids collectively encode for at least 50 variant genes. Further provided herein are oligonucleic acid libraries, wherein the 50 to 500 non-identical oligonucleic acids are attached to a surface of a structure and located within a discrete cluster. Further provided herein are oligonucleic acid libraries, wherein the 50 to 500 non-identical oligonucleic acids collectively encode at least 50 variant genes.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIGS. 1A-1D depict a process workflow for the synthesis of variant biological molecules incorporating a PCR mutagenesis step.

FIG. 2A-2D depict a process workflow for the generation of an oligonucleic acid comprising a nucleic acid sequence which differs from a reference oligonucleic acid sequence at a single predetermined codon site.

FIGS. 3A-3F depict an alternative workflow for the generation of a set of oligonucleic acid variants from a template oligonucleic acid, with each variant comprising a different nucleic acid sequence at a single codon position. Each variant oligonucleic acid encodes for a different amino acid at their single codon position, the different codons represented by X, Y, and Z.

FIG. 4A-4E depict a reference amino acid sequence (FIG. 4A) having a number of amino acids, each residue indicated by a single circle, and variant amino acid sequences (FIGS. 4B, 4C, 4D, & 4E) generated using methods described herein. The reference amino acid sequence and variant sequences are encoded by nucleic acids and variants thereof generated by processes described herein.

FIGS. 5A-5B depict a reference amino acid sequence (FIG. 5A, SEQ ID NO: 24) and a library of variant amino acid sequences (FIG. 5B, SEQ ID NOS 25-31, respectively, in order of appearance), each variant comprising a single residue variant (indicated by an “X”). The reference amino acid sequence and variant sequences are encoded by nucleic acids and variants thereof generated by processes described herein.

FIG. 6A-6B depict a reference amino acid sequence (FIG. 6A) and a library of variant amino acid sequences (FIG. 6B), each variant comprising two sites of single position variants. Each variant is indicated by differently patterned circles. The reference amino acid sequence and variant sequences are encoded by nucleic acids and variants thereof generated by processes described herein.

FIG. 7A-7B depict a reference amino acid sequence (FIG. 7A) and a library of variant amino acid sequences (FIG. 7B), each variant comprising a stretch of amino acids (indicated by a box around the circles), each stretch having three sites of position variants (encoding for histidine) differing in sequence from the reference amino acid sequence. The reference amino acid sequence and variant sequences are encoded by nucleic acids and variants thereof generated by processes described herein.

FIG. 8A-8B depict a reference amino acid sequence (FIG. 5A) and a library of variant amino acid sequences (FIG. 8B), each variant comprising two stretches of amino acid sequence (indicated by a box around the circles), each stretch having one site of single position variants (illustrated by the patterned circles) differing in sequence from reference amino acid sequence. The reference amino acid sequence and variant sequences are encoded by nucleic acids and variants thereof generated by processes described herein.

FIGS. 9A-9B depict a reference amino acid sequence (FIG. 9A) and a library of amino acid sequence variants (FIG. 9B), each variant comprising a stretch of amino acids (indicated by patterned circles), each stretch having a single site of multiple position variants differing in sequence from the reference amino acid sequence. In this illustration, 5 positions are varied where the first position has a 50/50 K/R ratio; the second position has a 50/25/25 V/US ratio, the third position has a 50/25/25 Y/R/D ratio, the fourth position has an equal ratio for all amino acids, and the fifth position has a 75/25 ratio for G/P. The reference amino acid sequence and variant sequences are encoded by nucleic acids and variants thereof generated by processes described herein.

FIG. 10 depicts a template oligonucleic acid encoding for an antibody having CDR1, CDR2, and CDR3 regions, where each CDR region comprises multiple sites for variation, each single site (indicated by a star) comprising a single position and/or stretch of multiple, consecutive positions interchangeable with any codon sequence different from the template oligonucleic acid sequence.

FIG. 11 depicts an exemplary number of variants produced by interchanging sections of two expression cassettes (e.g., promotors, open reading frames, and terminators) to generate a variant library of expression cassettes.

FIG. 12 presents a diagram of steps demonstrating an exemplary process workflow for gene synthesis as disclosed herein.

FIG. 13 illustrates an example of a computer system.

FIG. 14 is a block diagram illustrating an architecture of a computer system.

FIG. 15 is a diagram demonstrating a network configured to incorporate a plurality of computer systems, a plurality of cell phones and personal data assistants, and Network Attached Storage (NAS).

FIG. 16 is a block diagram of a multiprocessor computer system using a shared virtual address memory space.

FIG. 17 depicts a BioAnalyzer plot of PCR reaction products resolved by gel electrophoresis.

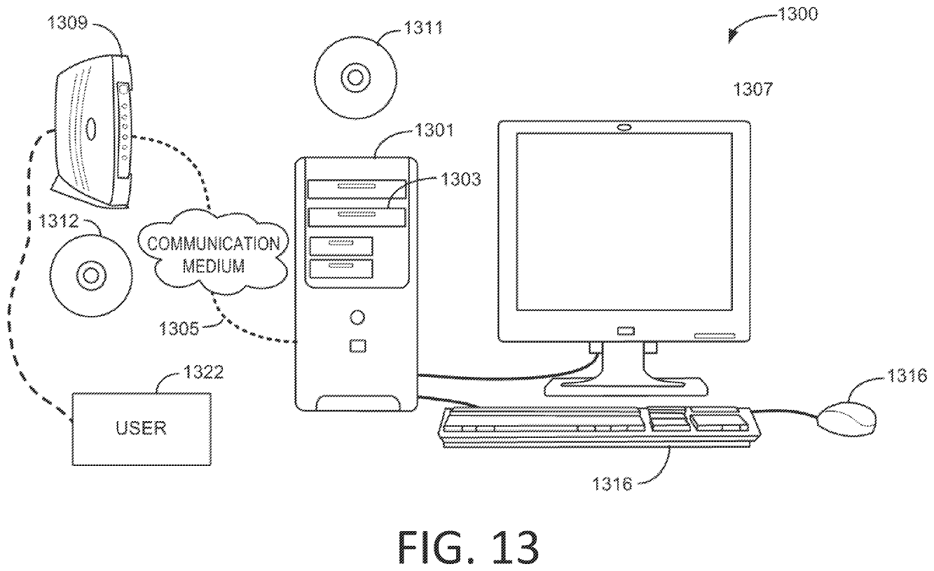

FIG. 13 depicts an electropherogram showing 96 sets of PCR products, each set of PCR products differing in sequence from a wild-type template nucleic acid at a single codon position, where the single codon position of each set is located at a different site in the wild-type template nucleic acid sequence. Each set of PCR products comprises 19 variant oligonucleic acids, each variant encoding for a different amino acid at their single codon position.

### DETAILED DESCRIPTION

The present disclosure employs, unless otherwise indicated, conventional molecular biology techniques, which are within the skill of the art. Unless defined otherwise, all technical and scientific terms used herein have the same meaning as is commonly understood by one of ordinary skill in the art.

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

Variant Library Synthesis

Methods described herein provide for synthesis of a library of oligonucleic acids each encoding for a predetermined variant of at least one predetermined reference nucleic acid sequence. In some cases, the predetermined reference sequence is nucleic acid sequence encoding for a protein, and the variant library comprises sequences encoding for variation of at least a single codon such that a plurality of different variants of a single residue in the subsequent protein encoded by the synthesized nucleic acid are generated by standard translation processes. The synthesized specific alterations in the nucleic acid sequence can be introduced by incorporating nucleotide changes into overlapping or blunt ended oligonucleic acid primers. Alternatively, a population of oligonucleic acids may collectively encode for a long nucleic acid (e.g., a gene) and variants thereof. In this arrangement, the population of oligonucleic acids can be hybridized and subject to standard molecular biology techniques to form the long nucleic acid (e.g., a gene) and variants thereof. When the long nucleic acid (e.g., a gene) and variants thereof are expressed in cells, a variant protein library is generated. Similarly, provided here are methods for synthesis of variant libraries encoding for RNA sequences (e.g., miRNA, shRNA, and mRNA) or DNA sequences (e.g., enhancer, promoter, UTR, and terminator regions). Also provided here are downstream applications for variants selected out of the libraries synthesized using methods describer here. Downstream applications include identification of variant nucleic acid or protein sequences with enhanced biologically relevant functions, e.g., biochemical affinity, enzymatic activity, changes in cellular activity, and for the treatment or prevention of a disease state.

Synthesis Followed by PCR Mutagenesis

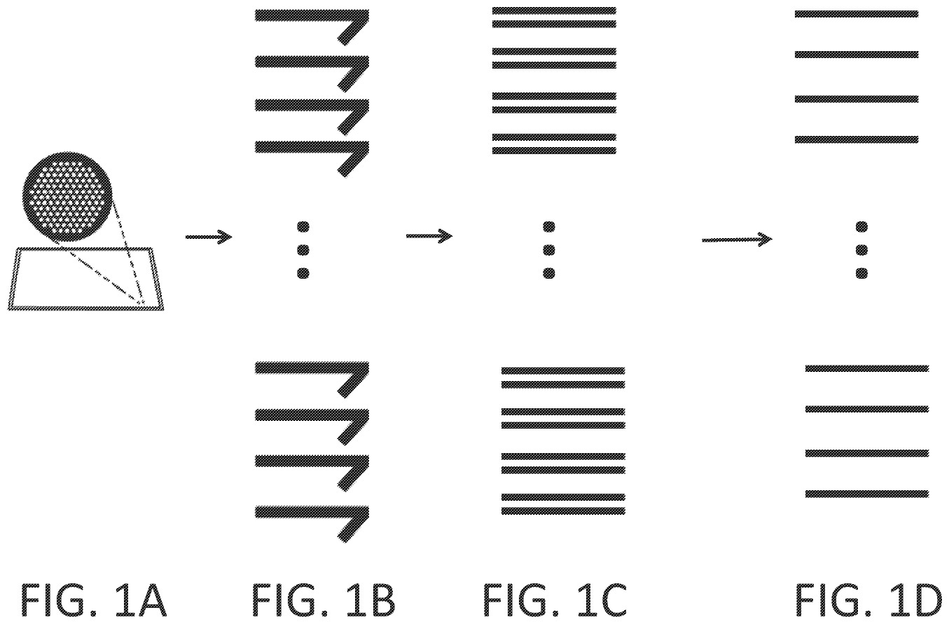

A first process for synthesis of a variant library of oligonucleic acids is for PCR mutagenesis methods. In this workflow, a plurality of oligonucleic acids are synthesized, wherein each oligonucleic acid encodes for a predetermined sequence which is a predetermined variant of a reference nucleic acid sequence. Referring to the figures, an exemplary workflow in depicted in FIGS. 1A-1D, wherein oligonucleic acids are generated on a surface. FIG. 1A depicts an expansion view of a single cluster of a surface with 121 loci. Each oligonucleic acid depicted in FIG. 1B is a primer that can be used for amplification from a reference nucleic acid sequence to produce a library of variant long nucleic acids, FIG. 1C. The library of variant long nucleic acids is then, optionally, subject to transcription and or translation to generate a variant RNA or protein library, FIG. 1D. In this exemplary illustration, a device having a substantially planar surface is used for de novo synthesis of oligonucleic acids is depicted, FIG. 1A. In some instances, the device comprises a cluster of loci, wherein each locus is a site for oligonucleic acid extension. In some instances, a single cluster comprises all the oligonucleic acid variants needed to generate a desired variant sequence library. In an alternative arrangement, a plate comprises a field of loci which are not segregated into clusters.

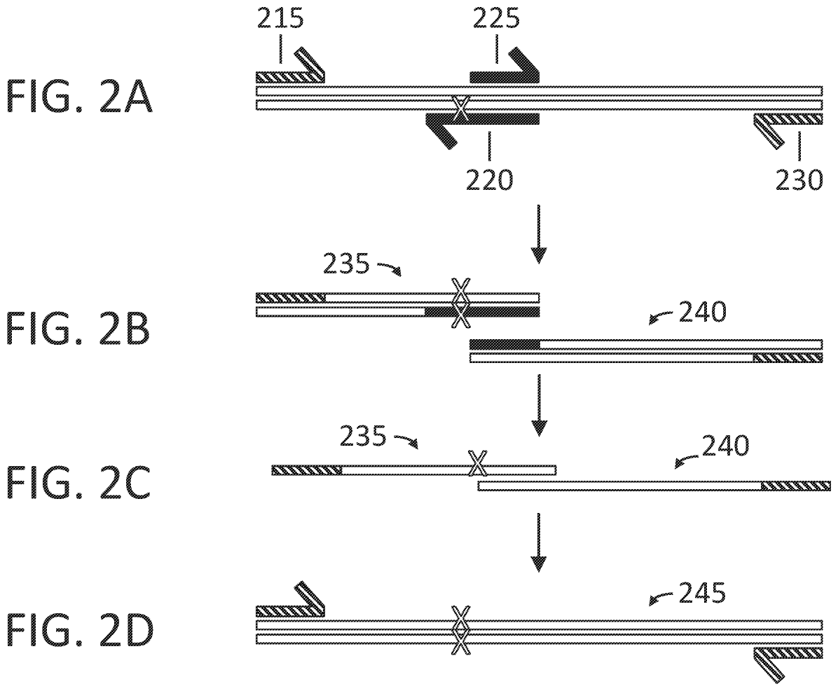

A de novo synthesized oligonucleic acid library described herein may comprise a plurality of oligonucleic acids, each with at least one variant sequence at first position, position “x”, and each variant oligonucleic acid is used as a primer in a first round of PCR to generate a first extension product. In this example, position “x” in a first oligonucleic acid 220 encodes for a variant codon sequence, i.e., one of 19 possible variants from a reference sequence. See FIG. 2A. A second oligonucleic acid 225 comprising sequence overlapping that of the first oligonucleic acid is also used as a primer in a separate round of PCR to generate a second extension product. In addition, outer primers 215, 230 may be used for amplification of fragment from a long nucleic acid sequence. The resultant amplification products are fragments of the long nucleic acid sequence 235, 240. See FIG. 2B. The fragments of the long nucleic acid sequence 235, 240 are then hybridized, and subject to an extension reaction to form a variant of the long nucleic acid 245. See FIG. 2C. The overlapping ends of the first and second extension products may serve as primer of a second round of PCR, thereby generating a third extension product (FIG. 2D) that contains the variant. To increase the yield, the variant of the long nucleic acid is amplified in a reaction including a DNA polymerase, amplification reagents, and the outer primers 215, 230. In some instances, the second oligonucleic acid comprises sequence adjacent to, but not including, the variant site. In an alternative arrangement, a first oligonucleic acid is generated that has region that overlaps with a second oligonucleic acid. In this scenario, the first oligonucleic acid is synthesized with variation at a single codon for up to 19 variants. The second oligonucleic acid does not comprise a variant sequence. Optionally, a first population comprises the first oligonucleic acid variants and additional oligonucleic acids encoding for variants at a different codon site. Alternatively, the first oligonucleic acid and the second oligonucleic acid may be designed for blunt end ligation.

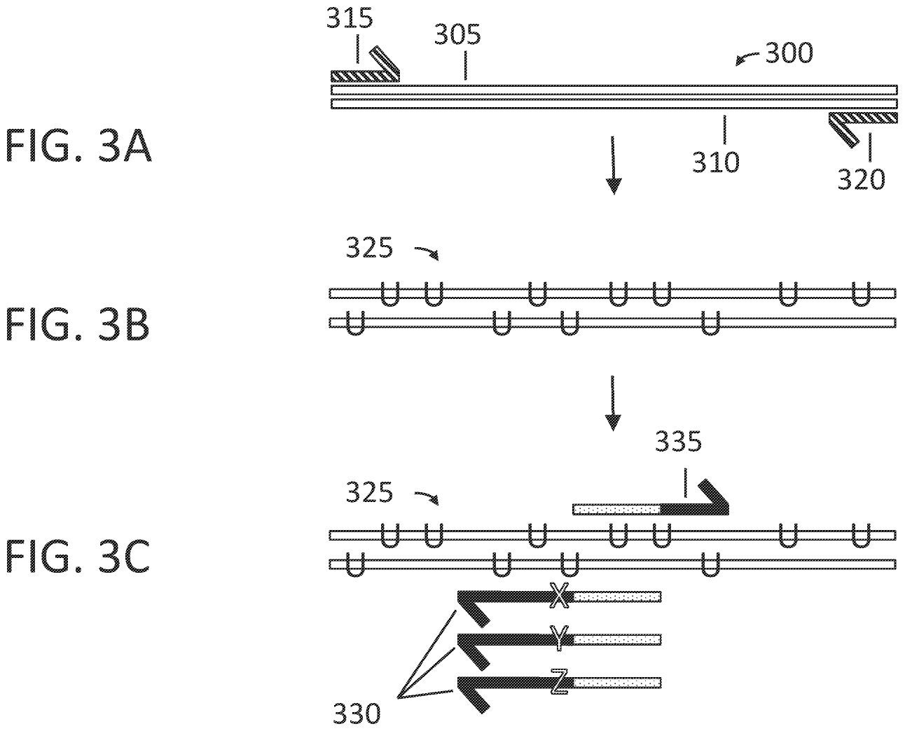

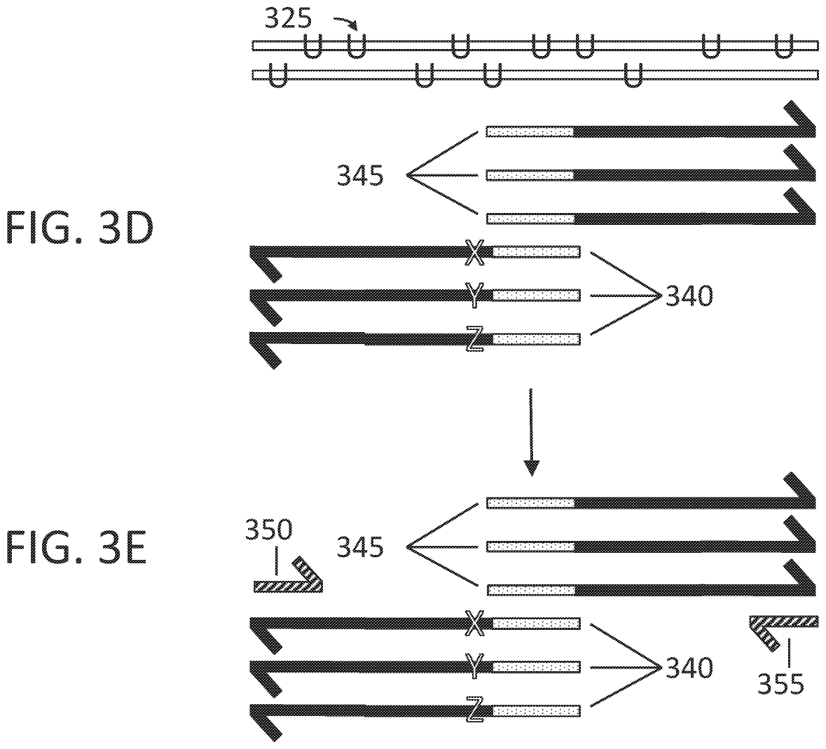

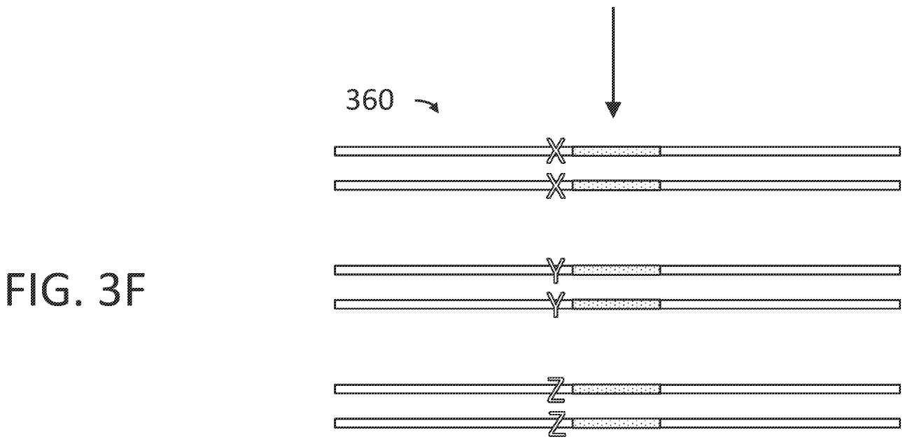

In alternative mutagenesis PCR method is depicted in FIGS. 3A-3F. In such a process, a template nucleic acid molecule 300 comprising a first and second strand 305, 310 is amplified in a PCR reaction containing a first primer 315 and a second primer 320 (FIG. 3A). The amplification reaction includes uracil as a nucleotide reagent. A uracil-labeled extension product 325 (FIG. 3B) is generated, optionally purified, and serves as a template for a subsequent PCR reaction using a first oligonucleic acid 335 and a plurality of second oligonucleic acid 330 to generate first extension products 340 and 345 (FIGS. 3C-3D). In this process, plurality of second oligonucleic acid 330 comprises oligonucleic acids encoding for variant sequences (denoted as X, Y, and Z, in FIG. 3C). The uracil-labeled template nucleic acid is digested by a uracil-specific excision reagent, e.g., USER digest available commercially from New England Biolabs. Variant 335 and different codons 330 with variants X, Y, and Z are added and a limited PCR step is performed to generate FIG. 3D. After the uracil-containing template is digested, the overlapping ends of the extension products serve to prime a PCR reaction with the first extension products 340 and 345 acting as primers in combination with a first outer primer 350 and a second outer primer 355, thereby generating a library of nucleic acid molecules 360 containing a plurality of variants X, Y, and Z at the variant site FIG. 3F.

De Novo Synthesis of a Population with Variant and Non-Variant Portions of a Long Nucleic Acid

In a second process for synthesis of a variant library, a surface is used for de novo synthesis of multiple fragments of a long nucleic acid, wherein at least one of the fragments is synthesized in multiple versions, each version being of a different variant sequence. In this arrangement, all of the fragments needed to assemble a library of variant long range nucleic acids are de novo synthesized. The synthesized fragments may have overlapping sequence such that, following synthesis, the fragment library is subject to hybridization. Following hybridization, an extension reaction may be performed to fill in any complementary gaps.

Alternatively, the synthesized fragments may be amplified with primers and then subject to either blunt end ligation or overlapping hybridization. In some instances, the device comprises a cluster of loci, wherein each locus is a site for oligonucleic acid extension. In some instances, a single cluster comprises all the oligonucleic acid variants and other fragment sequences of a predetermined long nucleic acid to generate a desired variant nucleic acid sequence library. The cluster may comprise about 50 to 500 loci. In some arrangements, a cluster comprises greater than 500 loci.

Each individual oligonucleic acid in the first oligonucleic acid population may be generated on a separate, individually addressable locus of a cluster. One oligonucleic acid variant may be represented by a plurality of individually addressable loci. Each variant in the first oligonucleic acid population may be represented 1, 2, 3, 4, 5, 6, 7, 8, 9, 10, or more times. In some instances, each variant in the first oligonucleic acid population is represented at 3 or less loci. In some instances, each variant in the first oligonucleic acid population is represented at two loci. In some instances, each variant in the first oligonucleic acid population is represented at only a single locus.

Methods are provided herein to generate nucleic acid libraries with reduced redundancy. In some instances, variant oligonucleotides may be generated without the need to synthesize the variant oligonucleotide more than 1 time to obtain the desired variant oligonucleotide. In some instances, the present disclosure provides methods to generate variant oligonucleotides without the need to synthesize the variant oligonucleotide more than 1, 2, 3, 4, 5 times, 6, 7, 8, 9, 10, or more times to generate the desired variant oligonucleotide.

Variant oligonucleotides may be generated without the need to synthesize the variant oligonucleotide at more than 1 discrete site to obtain the desired variant oligonucleotide. The present disclosure provides methods to generate variant oligonucleotides without the need to synthesize the variant oligonucleotide at more than 1 site, 2 sites, 3 sites, 4 sites, 5 sites, 6 sites, 7 sites, 8 sites, 9 sites, or 10 sites, to generate the desired variant oligonucleotide. In some instances, an oligonucleotide is synthesized in at most 6, 5, 4, 3, 2, or 1 discrete sites. The same oligonucleotide may be synthesized in 1, 2, or 3 discrete loci on a surface.

In some instances, the amount of loci representing a single variant oligonucleotide is a function of the amount of nucleic acid material required for downstream processing, e.g., an amplification reaction or cellular assay. In some instances, the amount of loci representing a single variant oligonucleotide is a function of the available loci in a single cluster.

Provided herein are methods for generation of a library of oligonucleic acids comprising variant oligonucleic acids differing at a plurality of sites in a reference nucleic acid. In such cases, each variant library is generated on an individually addressable locus within a cluster of loci. It will be understood that the number of variant sites represented by the oligonucleic acid library will be determined by the number of individually addressable loci in the cluster and the number of desired variants at each site. In some instances, each cluster comprises about 50 to 500 loci. In some instances, each cluster comprises 100 to 150 loci.

In an exemplary arrangement, 19 variants are represented at a variant site corresponding codons encoding for each of the 19 possible variant amino acids. In another exemplary case, 61 variants are represented at a variant site corresponding triplets encoding for each of the 19 possible variant amino acids. In a non-limiting example, a cluster comprises 121 individually addressable loci. In this example, an oligonucleic acid population comprises 6 replicates each of a single-site variant (6 replicates×1 variant site×19 variants=114 loci), 3 replicates each of a double-site variant (3 replicates×2 variant sites×19 variants=114 loci), or 2 replicates each of a triple-site variant (2 replicates×3 variant sites×19 variants=114 loci). In some instances, an oligonucleic acid population comprises variants at four, five, six or more than six variant sites.

Cadon Variation

Variant oligonucleic acid libraries described herein may comprise a plurality of oligonucleic acids, wherein each oligonucleic acid encodes for a variant codon sequence compared to a reference nucleic acid sequence. In some instances, each oligonucleic acid of a first oligonucleic acid population contains a variant at a single variant site. In some instances, the first oligonucleic acid population contains a plurality of variants at a single variant site such that the first oligonucleic acid population contains more than one variant at the same variant site. The first oligonucleic acid population may comprise oligonucleic acids collectively encoding multiple codon variants at the same variant site. The first oligonucleic acid population may comprise oligonucleic acids collectively encoding up to 19 or more codons at the same position. The first oligonucleic acid population may comprise oligonucleic acids collectively encoding up to 60 variant triplets at the same position, or the first oligonucleic acid population may comprise oligonucleic acids collectively encoding up to 61 different triplets of codons at the same position. Each variant may encode for a codon that results in a different amino acid during translation. Table 1 provides a listing of each codon possible (and the representative amino acid) for a variant site.

TABLE 1
List of codons and amino acids

| | One | Three | | | | | | |
|---|---|---|---|---|---|---|---|---|
| | letter | letter | | | | | | |
| Amino Acids | code | code | Codons | | | | | |
| Alanine | A | Ala | GCA | GCC | GCG | GCT | | |
| Cysteine | C | Cys | TGC | TGT | | | | |
| Aspartic acid | D | Asp | GAC | GAT | | | | |
| Glutamic acid | E | Glu | GAA | GAG | | | | |
| Phenylalanine | F | Phe | TTC | TTT | | | | |
| Glycine | G | Gly | GGA | GGC | GGG | GGT | | |
| Histidine | H | His | CAC | CAT | | | | |
| Isoleucine | I | Iso | ATA | ATC | ATT | | | |
| Lysine | K | Lys | AAA | AAG | | | | |
| Leucine | L | Leu | TTA | TTG | CTA | CTC | CTG | CTT |
| Methionine | M | Met | ATG | | | | | |
| Asparagine | N | Asn | AAC | AAT | | | | |
| Proline | P | Pro | CCA | CCC | CCG | CCT | | |
| Glutamine | Q | Gln | CAA | CAG | | | | |
| Arginine | R | Arg | AGA | AGG | CGA | CGC | CGG | CGT |
| Serine | S | Ser | AGC | AGT | TCA | TCC | TCG | TCT |

| | | | | | | |
|---|---|---|---|---|---|---|
| Threonine | T | Thr | ACA | ACC | ACG | ACT |
| Valine | V | Val | GTA | GTC | GTG | GTT |

| Tryptophan | W | Trp | TGG | |
|---|---|---|---|---|
| Tyrosine | Y | Tyr | TAC | TAT |

An oligonucleic acid population may comprise varied oligonucleic acids collectively encoding up to 20 codon variations at multiple positions. In such cases, each oligonucleic acid in the population comprises variation for codons at more than one position in the same oligonucleic acid. In some instances, each oligonucleic acid in the population comprises variation for codons at 1, 2, 3, 4, 5, 6, 7, 8, 9, 10, 11, 12, 13, 14, 15, 16, 17, 18, 19, 20 or more codons in a single oligonucleic acid. In some instances, each variant long nucleic acid comprises variation for 27, 28, 29, 30 or more codons in a single long nucleic acid. In some instances, the variant oligonucleic acid population comprises variation for codons at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more codons in a single oligonucleic acid. In some instances, the variant oligonucleic acid population comprises variation for codons in at least about 10, 20, 30, 40, 50, 60, 70, 80, 90, 100 or more codons in a single long nucleic acid.

Provided herein are processes where a second oligonucleic acid population is generated on a second cluster containing a plurality of individually addressable loci. The second oligonucleic acid population may comprise a plurality of second oligonucleic acids that are constant for each codon position (i.e., encode the same amino acid at each position). The second oligonucleic acid may overlap with at least a portion of the first oligonucleic acids. In some instances, the second oligonucleic acids do not contain the variant site represented on the first oligonucleic acids. Alternatively, the second oligonucleic acid population may comprise a plurality of second oligonucleic acids that variant for one or more codon positions.

Provided herein are methods for synthesizing a library of oligonucleic acids where a single population of oligonucleic acids is generated comprising variants at multiple codon positions. A first oligonucleic acid population may be generated on a first cluster containing a plurality of individually addressable loci. In such cases, the first oligonucleic acid population comprises variants at a different codon positions. In some instances, the different sites are consecutive (i.e., encoding consecutive amino acids). A first oligonucleotide acid population may comprise varied oligonucleic acids collectively encoding up to 19 codon variants at the same, or additional variant site. A first oligonucleotide acid population may include a plurality of first oligonucleic acids that contains up to 19 variants at position x, up to 19 variants at position y, and up to 19 variants at position z. In such an arrangement, each variant encodes a different amino acid such that up to 19 amino acid variants are encoded at each of the different variant sites. In an additional instance, a second oligonucleic acid population is generated on a second cluster containing a plurality of individually addressable loci. The second oligonucleic acid population may comprise a plurality of second oligonucleic acids that are constant for each codon position (i.e., encode the same amino acid at each position). The second oligonucleic acids may overlap with at least a portion of the first oligonucleic acids. The second oligonucleic acids may not contain the variant site represented on the first oligonucleic acids.

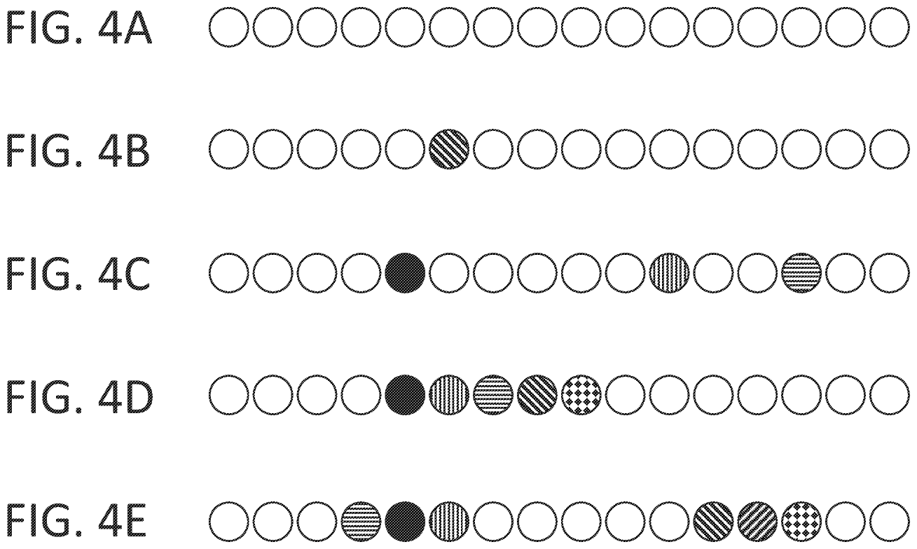

Variant nucleic acid libraries generated by processes described herein provide for the generation of variant protein libraries. In a first exemplary arrangement, a template oligonucleic acid encodes for sequence that, when transcribed and translated, results in a reference amino acid sequence (FIG. 4A) having a number of codon positions, indicated by a single circle. Oligonucleic acid variants of the template can be generated using methods described herein. In some instances, a single variant is present in the oligonucleic acid, resulting in a single amino acid sequence (FIG. 4B). In some instances, more than one variant is present in the oligonucleic acid, wherein the variants are separated by one or more codons, resulting in a protein with spacing between variant residues (FIG. 4C). In some instances, more than one variant is present in the oligonucleic acid, wherein the variants are sequential and adjacent or consecutive to one another, resulting in spaced variant stretches of residues (FIG. 4D). In some instances, two stretches of variants are present in the oligonucleic acid, wherein each stretch of variants comprises sequential and adjacent or consecutive variants (FIG. 4E).

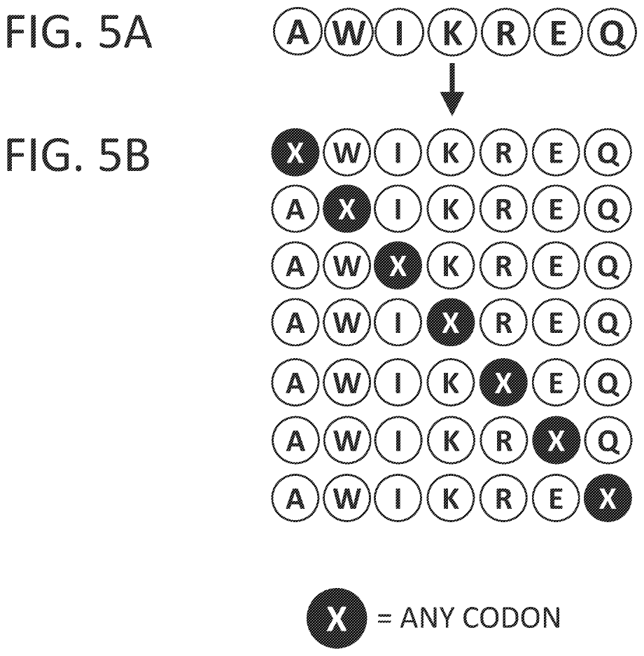

Provided herein are methods to generate a library of oligonucleic acid variants, wherein each variant comprises a single position codon variant. In one instance, a template oligonucleic acid has a number of codon positions wherein exemplary amino acid residues are indicated by circles with their respective one letter code protein codon, FIG. 5A. FIG. 5B depicts a library of amino acid variants encoded by a library of variant nuclei acids, wherein each variant comprises a single position variant, indicated by an “X”, of located at a different single site. A first position variant has any codon to replace alanine, a second variant with any codon encoded by the library of variant nuclei acids to replace tryptophan, a third variant with any codon to replace isoleucine, a fourth variant with any codon to replace lysine, a fifth variant with any codon to replace arginine, a sixth variant with any codon to replace glutamic acid, and a seventh variant with any codon to replace glutamine. When all or less than all codon variants are encoded by the variant nucleic acid library, a resulting a corresponding population of amino acid sequence variants is generated following protein expression (i.e., standard cellular events of DNA transcription followed by translation and processing events).

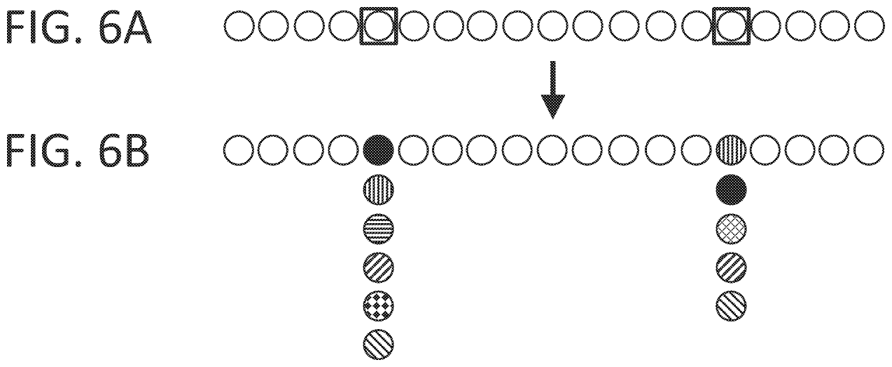

In some arrangements, a library is generated with multiple sites of single position variants. As depicted in FIG. 6A, a wild-type template is provided. FIG. 6B depicts the resultant amino acid sequence with two sites of single position codon variants, wherein each codon variant encoding for a different amino acid is indicated by differently patterned circles.

Provided herein are methods to generate a library having a stretch of multiple site, single position variants. Each stretch of oligonucleic acid may have 1, 2, 3, 4, 5, or more variants. Each stretch of oligonucleic acid may have at least 1 variants. Each stretch of oligonucleic acid may have at least 2 variants. Each stretch of oligonucleic acid may have at least 3 variants. For example, a stretch of 5 oligonucleic acids may have 1 variant. A stretch of 5 oligonucleic acids may have 2 variants. A stretch of 5 oligonucleic acids may have 3 variants. A stretch of 5 oligonucleic acids may have 4 variants. For example, a stretch of 4 oligonucleic acids may have 1 variant. A stretch of 4 oligonucleic acids may have 2 variants. A stretch of 4 oligonucleic acids may have 3 variants. A stretch of 4 oligonucleic acids may have 4 variants.

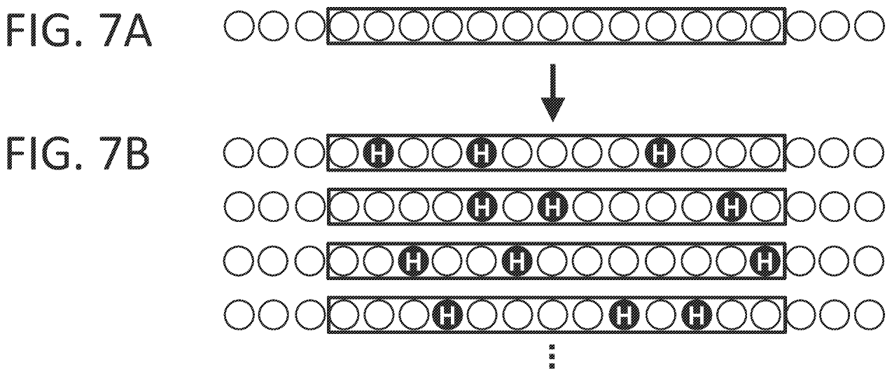

In some instances, single position variants may all encode for the same amino acid, e.g. a histidine. As depicted in FIG. 7A, a reference amino acid sequence is provided. In this arrangement, a stretch of an oligonucleic acid encodes for multiple sites of single position variants and, when expressed, results in an amino acid sequence having all single position variants encoding for a histidine, FIG. 7B. In some embodiments, a variant library synthesized by methods described herein does not encode for more than 4 histidine residues in a resultant amino acid sequence.

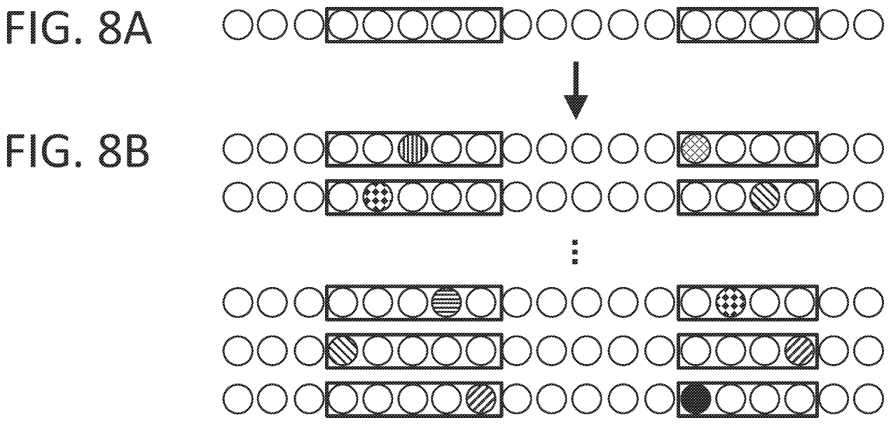

In some instances, a variant library of nucleic acids generated by methods described herein provides for expression of amino acid sequences have separate stretches of variation. A template amino acid sequence is depicted in FIG. 8A. A stretch of oligonucleic acids may have only 1 variant codon in two stretches and, when expressed, result in an amino acid sequence depicted in FIG. 8B. Variants are depicted in FIG. 8B by the differently patterned circles to indicate variation in amino acids are different position in a single stretch.

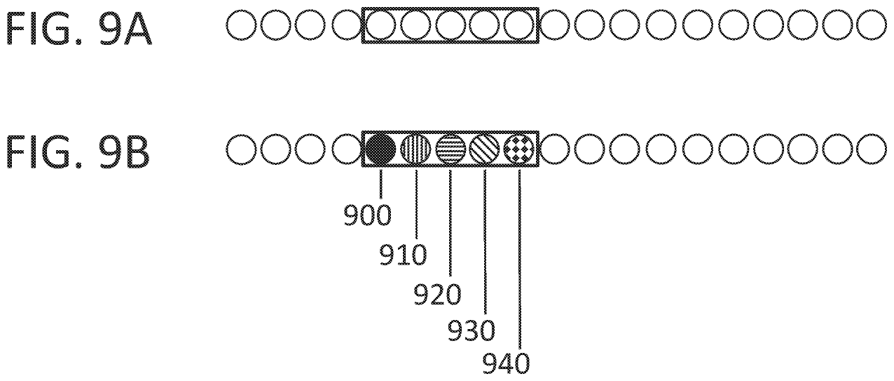

Provided herein are methods and devices to synthesize oligonucleic acid libraries with 1, 2, 3, or more codon variants, wherein the variant for each site is selectively controlled. The ratio of two amino acids for a single site variant may be about 1:100, 1:50, 1:10, 1:5, 1:3, 1:2, 1:1. The ratio of three amino acids for a single site variant may be about 1:1:100, 1:1:50, 1:1:20, 1:1:10, 1:1:5, 1:1:3, 1:1:2, 1:1:1, 1:10:10, 1:5:5, 1:3:3, or 1:2:2. FIG. 9A depicts a wild-type reference amino acid sequence encoded by a wild-type nucleic acid sequence. FIG. 9B depicts a library of amino acid variants, wherein each variant comprising a stretch of sequence (indicated by the patterned circles), wherein each position may have a certain ratio of amino acids in the resultant variant protein library. The resultant variant protein library is encoded by a variant nucleic acid library generated by methods described herein. In this illustration, 5 positions are varied: the first position 900 has a 50/50 K/R ratio; the second position 910 has a 50/25/25 V/US ratio, the third position 920 has a 50/25/25 Y/R/D ratio, the fourth position 930 has an equal ratio for all 20 amino acids, and the fifth position 940 has a 75/25 ratio for G/P. The ratios described herein are exemplary only.

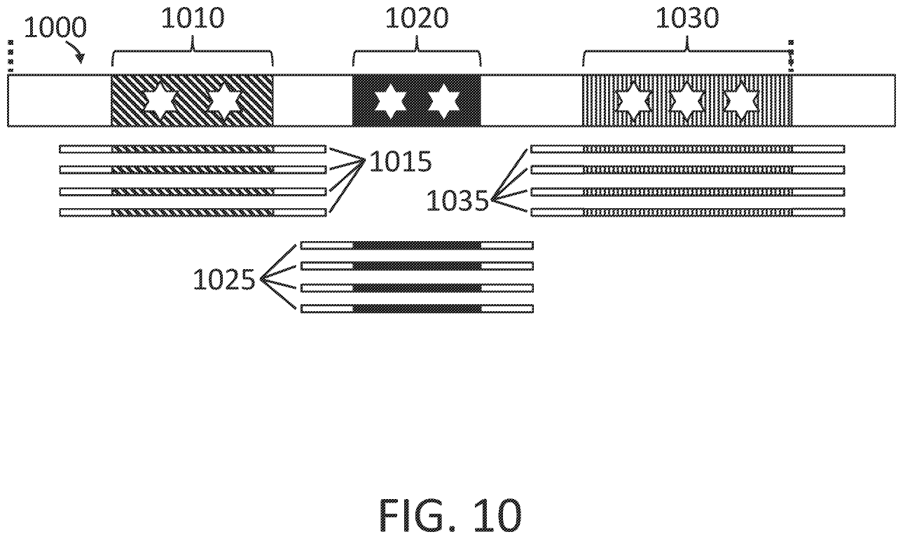

In some instances, a synthesized variant library is generated which encodes for nucleic acid sequence that is ultimately translated into amino acid sequence of a protein. Exemplary into amino acid sequence includes those encoding for small peptides as well as at least a portion of large peptides, e.g., antibody sequence. In some instances, the oligonucleic acids synthesized each encode for a variant codon in a portion of an antibody sequence. Exemplary antibody sequence for which the portion of variant synthesized oligonucleic acid encodes includes the antigen-binding or variable region thereof, or a fragment thereof. Examples antibody fragments for which the oligonucleic acids described herein encode a portion of include, without limitation, Fab, Fab′, F(ab′)2 and Fv fragments, diabodies, linear antibodies, single-chain antibody molecules, and multispecific antibodies formed from antibody fragments. Examples antibody regions for which the oligonucleic acids described herein encode a portion of include, without limitation, Fc region, Fab region, variable region of the Fab region, constant region of the Fab region, variable domain of the heavy chain or light chain (V_{H} or V_{L}), or specific complementarity-determining regions (CDRs) of V_{H} or V_{L}. Variant libraries generated by methods disclosed herein can result in variation of one or more of the antibody regions described herein. In one exemplary process, a variant library is generated for nucleic acids encoding for a several CDRs. See FIG. 10. A template nucleic acid encoding for an antibody having CDR1 1010, CDR2 1020, and CDR3 1030 regions, is modified by methods described herein, where each CDR region comprises multiple sites for variation. Variations for each of 3 CDRs in a single variable domain of a heavy chain or light chain 1015, 1025, and 1035 are generated. Each site, indicated by a star, may comprise a single position, a stretch of multiple, consecutive positions, or both, that are interchangeable with any codon sequence different from the template oligonucleic acid sequence. Diversity of variant libraries may dramatically increase using methods provided herein, with up to ˜10¹⁰ diversity, or more.

Variation in Expression Cassettes

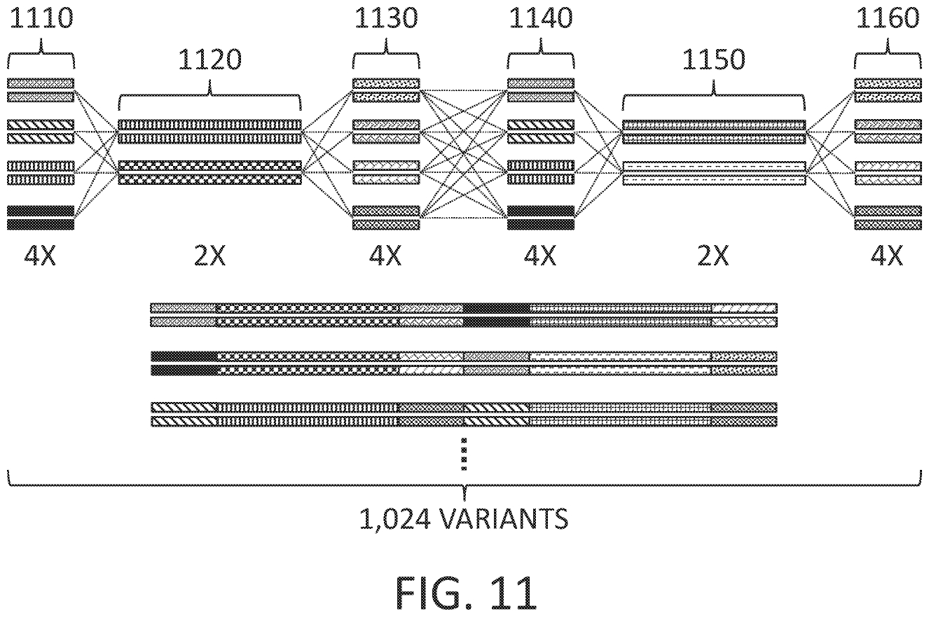

In some instances, a synthesized variant library is generated which encodes for a portion of an expression construct. Exemplary portions of an expression construct include the promoter, open reading frame, and termination region. In some instances, the expression construct encodes for one, two, three or more expression cassettes. An oligonucleotide library may be generated, encoding for codon variation at a single site or multiple sites separate regions that make up portions of an expression construct cassette, as depicted in FIG. 11. To generate a two construct expressing cassette, variant oligonucleic acids were synthesized encoding at least a portion of a variant sequence of a first promoter 1110, first open reading frame 1120, first terminator 1130, second promoter 1140, second open reading frame 1150, or second terminator sequence 1160. After rounds of amplification, as described in previous examples, a library of 1,024 expression constructs was generated. FIG. 11 provides but one example arrangement. In some instances, additional regulator sequences, such as untranslated regulatory region (UTR) or an enhancer region, is are also included in an expression cassette referred to herein. An expression cassette may comprise 1, 2, 3, 4, 5, 6, 7, 8, 9, 10 or more components for which variant sequences are generated by methods described herein. In some instances, the expression construct comprises more than one gene in a multicistronic vector. In one example, the synthesized DNA oligonucleic acids are inserted into viral vectors (e.g., a lentivirus) and then packaged for transduction into cells, or non-viral vectors for transfer into cells, followed by screening and analysis.

Expression vectors for inserting nucleic acids disclosed herein comprise eukaryotic (e.g., bacterial and fungal) and prokaryotic (e.g., mammalian, plant and insect expression vectors). Exemplary expression vectors include, without limitation, mammalian expression vectors: pSF-CMV-NEO-NH2-PPT-3×FLAG, pSF-CMV-NEO—COOH-3×FLAG, pSF-CMV-PURO-NH2-GST-TEV, pSF-OXB20-COOH-TEV-FLAG®-6His, pCEP4 pDEST27, pSF-CMV-Ub-KrYFP, pSF-CMV-FMDV-daGFP, pEF1a-mCherry-N1 Vector, pEFIa-tdTomato Vector, pSF-CMV-FMDV-Hygro, pSF-CMV-PGK-Puro, pMCP-tag(m), and pSF-CMV-PURO-NH2-CMYC; bacterial expression vectors: pSF-OXB20-BetaGal, pSF-OXB20-Fluc, pSF-OXB20, and pSF-Tac; plant expression vectors: pRI 101-AN DNA and pCambia2301; and yeast expression vectors: pTYB21 and pKLAC2, and insect vectors: pAc5.1/V5-His A and pDEST8. Exemplary cells include without limitation, prokaryotic and eukaryotic cells. Exemplary eukaryotic cells include, without limitation, animal, plant, and fungal cells. Exemplary animal cells include, without limitation, insect, fish and mammalian cells. Exemplary mammalian cells include mouse, human, and primate cells. Nucleic acids synthesized by methods described herein may be transferred into cells done by various methods known in the art, including, without limitation, transfection, transduction, and electroporation. Exemplary cellular functions tested include, without limitation, changes in cellular proliferation, migration/adhesion, metabolic, and cell-signaling activity.

Highly Parallel Nucleic Acid Synthesis

Provided herein is a platform approach utilizing miniaturization, parallelization, and vertical integration of the end-to-end process from oligonucleic acid synthesis to gene assembly within nanowells on silicon to create a revolutionary synthesis platform. Devices described herein provide, with the same footprint as a 96-well plate, a silicon synthesis platform is capable of increasing throughput by a factor of up to 1,000 or more compared to traditional synthesis methods, with production of up to approximately 1,000,000 or more oligonucleic acids, or 10,000 or more genes in a single highly-parallelized run.

With the advent of next-generation sequencing, high resolution genomic data has become an important factor for studies that delve into the biological roles of various genes in both normal biology and disease pathogenesis. At the core of this research is the central dogma of molecular biology and the concept of “residue-by-residue transfer of sequential information.” Genomic information encoded in the DNA is transcribed into a message that is then translated into the protein that is the active product within a given biological pathway.

Another exciting area of study is on the discovery, development and manufacturing of therapeutic molecules focused on a highly-specific cellular target. High diversity DNA sequence libraries are at the core of development pipelines for targeted therapeutics. Gene mutants are used to express proteins in a design, build, and test protein engineering cycle that ideally culminates in an optimized gene for high expression of a protein with high affinity for its therapeutic target. As an example, consider the binding pocket of a receptor. The ability to test all sequence permutations of all residues within the binding pocket simultaneously will allow for a thorough exploration, increasing chances of success. Saturation mutagenesis, in which a researcher attempts to generate all possible mutations at a specific site within the receptor, represents one approach to this development challenge. Though costly and time and labor-intensive, it enables each variant to be introduced into each position. In contrast, combinatorial mutagenesis, where a few selected positions or short stretch of DNA may be modified extensively, generates an incomplete repertoire of variants with biased representation.

To accelerate the drug development pipeline, a library with the desired variants available at the intended frequency in the right position available for testing—in other words, a precision library, enables reduced costs as well as turnaround time for screening. Provided herein are methods for synthesizing oligonucleic acid synthetic variant libraries which provide for precise introduction of each intended variant at the desired frequency. To the end user, this translates to the ability to not only thoroughly sample sequence space but also be able to query these hypotheses in an efficient manner, reducing cost and screening time. Genome-wide editing can elucidate important pathways, libraries where each variant and sequence permutation can be tested for optimal functionality, and thousands of genes can be used to reconstruct entire pathways and genomes to re-engineer biological systems for drug discovery.

In a first example, a drug itself can is optimized using methods described herein. For example, to improve a specified function of an antibody, a variant oligonucleic acid library encoding for a portion of the antibody is designed and synthesized. A variant nucleic acid library for the antibody can then be generated by processes described herein (e.g., PCR mutagenesis followed by insertion into a vector). The antibody is then expressed in a production cell line and screened for enhanced activity. Example screens include examining modulation in binding affinity to an antigen, stability, or effector function (e.g., ADCC, complement, or apoptosis). Exemplary regions to optimize the antibody include, without limitation, the Fc region, Fab region, variable region of the Fab region, constant region of the Fab region, variable domain of the heavy chain or light chain (V_{H} or V_{L}), and specific complementarity-determining regions (CDRs) of V_{H} or V_{L}.

Alternatively, the molecule to optimize is a receptor binding epitope for use as an activating agent or competitive inhibitor. Subsequent to synthesis of a variant library of nucleic acids, the variant library of nucleic acids may be inserted into vector sequence and then expressed in cells. The receptor antigen may be expressed in cells (e.g., insect, mammalian or bacterial) and then purified, or it may be expressed in cells (e.g., mammalian) to examine functional consequence from variation the sequence. Functional consequences include, without limitation, a change in the proteins expression, binding affinity and stability. Cellular functional consequence include, without limitation, a change in reproduction, growth, adhesion, death, migration, energy production, oxygen utilization, metabolic activity, cell signaling, aging, response to free radical damage, or any combination thereof. In some embodiments, the type of protein selected for optimization is an enzyme, transporter proteins, G-protein coupled receptors, voltage-gated ion channels, transcription factors, polymerases, adaptor proteins (proteins without enzymatic activity the serve to bring two other proteins together), and cytoskeletal proteins. Exemplary types of enzyme include, without limitation, signalling enzymes (such as protein kinases, protein phosphatases, phosphodiesterases, histone deacetylases, and GTPases).

Provided herein are variant nucleic acid libraries comprising variants for molecules involved in an entire pathway or an entire genome. Exemplary pathways include, without limitation a metabolic, cell death, cell cycle progression, immune cell activation, inflammatory response, angiogenesis, lymphogenesis, hypoxia and oxidative stress response, or cell adhesion/migration pathway. Exemplary proteins in a cell death pathway include, without limitation, Fas, Cadd, Caspase 3, Caspase 6, Caspase 8, Caspase 9, Caspase 10, lAP, TNFR1, TNF, TNFR2, NF-kB, TRAFs, ASK, BAD, and Akt. Exemplary proteins in a cell cycle pathway include, without limitation, NFkB, E2F, Rb, p53, p21, cyclin A, cyclin B, cyclin D, cyclin E, and cdc 25. Exemplary proteins in a cell migration pathway include, without limitation, Ras, Raf, PLC, cofilin, MEK, ERK, MLP, LIMK, ROCK, RhoA, Src, Rac, Myosin II, ARP2/3, MAPK, PIP2, integrins, talin, kindlin, migfilin and filamin.

Nucleic acid libraries synthesized by methods described herein may be expressed in various cell types. Exemplary cell types include prokaryotes (e.g., bacteria and fungi) and eukaryotes (e.g., plants and animals). Exemplary animals include, without limitation, mice, rabbits, primates, fish, and insects. Exemplary plants include, without limitation, a monocot and dicot. Exemplary plants also include, without limitation, microalgae, kelp, cyanobacteria, and green, brown and red algae, wheat, tobacco, and corn, rice, cotton, vegetables, and fruit.

Nucleic acid libraries synthesized by methods described herein may be expressed in various cells associated with a disease state. Cells associated with a disease state include cell lines, tissue samples, primary cells from a subject, cultured cells expanded from a subject, or cells in a model system. Exemplary model systems include, without limitation, plant and animal models of a disease state.

Nucleic acid libraries synthesized by methods described herein may be expressed in various cell types assess a change in cellular activity. Exemplary cellular activities include, without limitation, proliferation, cycle progression, cell death, adhesion, migration, reproduction, cell signaling, energy production, oxygen utilization, metabolic activity, and aging, response to free radical damage, or any combination thereof.

To identify a variant molecule associated with prevention, reduction or treatment of a disease state, a variant nucleic acid library described herein is expressed in a cell associated with a disease state, or one in which a cell a disease state can be induced. In some instances, an agent is used to induce a disease state in cells. Exemplary tools for disease state induction include, without limitation, a Cre/Lox recombination system, LPS inflammation induction, and streptozotocin to induce hypoglycemia. The cells associated with a disease state may be cells from a model system or cultured cells, as well as cells from a subject having a particular disease condition. Exemplary disease conditions include a bacterial, fungal, viral, autoimmune, or proliferative disorder (e.g., cancer). In some instances, the variant nucleic acid library is expressed in the model system, cell line, or primary cells derived from a subject, and screened for changes in at least one cellular activity. Exemplary cellular activities include, without limitation, proliferation, cycle progression, cell death, adhesion, migration, reproduction, cell signaling, energy production, oxygen utilization, metabolic activity, and aging, response to free radical damage, or any combination thereof.

Substrates

Provided herein are substrates comprising a plurality of clusters, wherein each cluster comprises a plurality of loci that support the attachment and synthesis of oligonucleic acids. The term “locus” as used herein refers to a discrete region on a structure which provides support for oligonucleotides encoding for a single predetermined sequence to extend from the surface. In some instances, a locus is on a two dimensional surface, e.g., a substantially planar surface. In some instances, a locus refers to a discrete raised or lowered site on a surface e.g., a well, microwell, channel, or post. In some instances, a surface of a locus comprises a material that is actively functionalized to attach to at least one nucleotide for oligonucleic acid synthesis, or preferably, a population of identical nucleotides for synthesis of a population of oligonucleic acids. In some instances, oligonucleic acid refers to a population of oligonucleic acids encoding for the same nucleic acid sequence. In some instances, a surface of a device is inclusive of one or a plurality of surfaces of a substrate.

Average error rates for oligonucleic acids synthesized within a library using the systems and methods provided may be less than 1 in 1000, less than 1 in 1250, less than 1 in 1500, less than 1 in 2000, less than 1 in 3000 or less often. In some instances, average error rates for oligonucleic acids synthesized within a library using the systems and methods provided are less than 1/500, 1/600, 1/700, 1/800, 1/900, 1/1000, 1/1100, 1/1200, 1/1250, 1/1300, 1/1400, 1/1500, 1/1600, 1/1700, 1/1800, 1/1900, 1/2000, 1/3000, or less. In some instances, average error rates for oligonucleic acids synthesized within a library using the systems and methods provided are less than 1/1000.

In some instances, aggregate error rates for oligonucleic acids synthesized within a library using the systems and methods provided are less than 1/500, 1/600, 1/700, 1/800, 1/900, 1/1000, 1/1100, 1/1200, 1/1250, 1/1300, 1/1400, 1/1500, 1/1600, 1/1700, 1/1800, 1/1900, 1/2000, 1/3000, or less compared to the predetermined sequences. In some instances, aggregate error rates for oligonucleic acids synthesized within a library using the systems and methods provided are less than 1/500, 1/600, 1/700, 1/800, 1/900, or 1/1000. In some instances, aggregate error rates for oligonucleic acids synthesized within a library using the systems and methods provided are less than 1/1000.

In some instances, an error correction enzyme may be used for oligonucleic acids synthesized within a library using the systems and methods provided can use. In some instances, aggregate error rates for oligonucleic acids with error correction can be less than 1/500, 1/600, 1/700, 1/800, 1/900, 1/1000, 1/1100, 1/1200, 1/1300, 1/1400, 1/1500, 1/1600, 1/1700, 1/1800, 1/1900, 1/2000, 1/3000, or less compared to the predetermined sequences. In some instances, aggregate error rates with error correction for oligonucleic acids synthesized within a library using the systems and methods provided can be less than 1/500, 1/600, 1/700, 1/800, 1/900, or 1/1000. In some instances, aggregate error rates with error correction for oligonucleic acids synthesized within a library using the systems and methods provided can be less than 1/1000.

Error rate may limit the value of gene synthesis for the production of libraries of gene variants. With an error rate of 1/300, about 0.7% of the clones in a 1500 base pair gene will be correct. As most of the errors from oligonucleotide synthesis result in frame-shift mutations, over 99% of the clones in such a library will not produce a full-length protein. Reducing the error rate by 75% would increase the fraction of clones that are correct by a factor of 40. The methods and compositions of the disclosure allow for fast de novo synthesis of large oligonucleotide and gene libraries with error rates that are lower than commonly observed gene synthesis methods both due to the improved quality of synthesis and the applicability of error correction methods that are enabled in a massively parallel and time-efficient manner. Accordingly, libraries may be synthesized with base insertion, deletion, substitution, or total error rates that are under 1/300, 1/400, 1/500, 1/600, 1/700, 1/800, 1/900, 1/1000, 1/1250, 1/1500, 1/2000, 1/2500, 1/3000, 1/4000, 1/5000, 1/6000, 1/7000, 1/8000, 1/9000, 1/10000, 1/12000, 1/15000, 1/20000, 1/25000, 1/30000, 1/40000, 1/50000, 1/60000, 1/70000, 1/80000, 1/90000, 1/100000, 1/125000, 1/150000, 1/200000, 1/300000, 1/400000, 1/500000, 1/600000, 1/700000, 1/800000, 1/900000, 1/1000000, or less, across the library, or across more than 80%, 85%, 90%, 93%, 95%, 96%, 97%, 98%, 99%, 99.5%, 99.8%, 99.9%, 99.95%, 99.98%, 99.99%, or more of the library. The methods and compositions of the disclosure further relate to large synthetic oligonucleotide and gene libraries with low error rates associated with at least 30%, 40%, 50%, 60%, 70%, 75%, 80%, 85%, 90%, 93%, 95%, 96%, 97%, 98%, 99%, 99.5%, 99.8%, 99.9%, 99.95%, 99.98%, 99.99%, or more of the oligonucleotides or genes in at least a subset of the library to relate to error free sequences in comparison to a predetermined/preselected sequence. In some instances, at least 30%, 40%, 50%, 60%, 70%, 75%, 80%, 85%, 90%, 93%, 95%, 96%, 97%, 98%, 99%, 99.5%, 99.8%, 99.9%, 99.95%, 99.98%, 99.99%, or more of the oligonucleotides or genes in an isolated volume within the library have the same sequence. In some instances, at least 30%, 40%, 50%, 60%, 70%, 75%, 80%, 85%, 90%, 93%, 95%, 96%, 97%, 98%, 99%, 99.5%, 99.8%, 99.9%, 99.95%, 99.98%, 99.99%, or more of any oligonucleotides or genes related with more than 95%, 96%, 97%, 98%, 99%, 99.5%, 99.6%, 99.7%, 99.8%, 99.9% or more similarity or identity have the same sequence. In some instances, the error rate related to a specified locus on an oligonucleotide or gene is optimized. Thus, a given locus or a plurality of selected loci of one or more oligonucleotides or genes as part of a large library may each have an error rate that is less than 1/300, 1/400, 1/500, 1/600, 1/700, 1/800, 1/900, 1/1000, 1/1250, 1/1500, 1/2000, 1/2500, 1/3000, 1/4000, 1/5000, 1/6000, 1/7000, 1/8000, 1/9000, 1/10000, 1/12000, 1/15000, 1/20000, 1/25000, 1/30000, 1/40000, 1/50000, 1/60000, 1/70000, 1/80000, 1/90000, 1/100000, 1/125000, 1/150000, 1/200000, 1/300000, 1/400000, 1/500000, 1/600000, 1/700000, 1/800000, 1/900000, 1/1000000, or less. In various instances, such error optimized loci may comprise at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500, 600, 700, 800, 900, 1000, 1500, 2000, 2500, 3000, 4000, 5000, 6000, 7000, 8000, 9000, 10000, 30000, 50000, 75000, 100000, 500000, 1000000, 2000000, 3000000 or more loci. The error optimized loci may be distributed to at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500, 600, 700, 800, 900, 1000, 1500, 2000, 2500, 3000, 4000, 5000, 6000, 7000, 8000, 9000, 10000, 30000, 75000, 100000, 500000, 1000000, 2000000, 3000000 or more oligonucleotides or genes.

The error rates can be achieved with or without error correction. The error rates can be achieved across the library, or across more than 80%, 85%, 90%, 93%, 95%, 96%, 97%, 98%, 99%, 99.5%, 99.8%, 99.9%, 99.95%, 99.98%, 99.99⁰%, or more of the library.

Provided herein are structures that may comprise a surface that supports the synthesis of a plurality of oligonucleic acids having different predetermined sequences at addressable locations on a common support. In some instances, a device provides support for the synthesis of more than 2,000; 5,000; 10,000; 20,000; 30,000; 50,000; 75,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more non-identical oligonucleic acids. In some instances, the device provides support for the synthesis of more than 2,000; 5,000; 10,000; 20,000; 30,000; 50,000; 75,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more oligonucleic acids encoding for distinct sequences. In some instances, at least a portion of the oligonucleic acids have an identical sequence or are configured to be synthesized with an identical sequence.

Provided herein are methods and devices for manufacture and growth of oligonucleic acids about 5, 10, 20, 30, 40, 50, 60, 70, 80, 90, 100, 125, 150, 175, 200, 225, 250, 275, 300, 325, 350, 375, 400, 425, 450, 475, 500, 600, 700, 800, 900, 1000, 1100, 1200, 1300, 1400, 1500, 1600, 1700, 1800, 1900, or 2000 bases in length. In some instances, the length of the oligonucleic acid formed is about 5, 10, 20, 30, 40, 50, 60, 70, 80, 90, 100, 125, 150, 175, 200, or 225 bases in length. An oligonucleic acid may be at least 5, 10, 20, 30, 40, 50, 60, 70, 80, 90, or 100 bases in length. An oligonucleic acid may be from 10 to 225 bases in length, from 12 to 100 bases in length, from 20 to 150 bases in length, from 20 to 130 bases in length, or from 30 to 100 bases in length.

In some instances, oligonucleic acids are synthesized on distinct loci of a substrate, wherein each locus supports the synthesis of a population of oligonucleic acids. In some instances, each locus supports the synthesis of a population of oligonucleic acids having a different sequence than a population of oligonucleic acids grown on another locus. In some instances, the loci of a device are located within a plurality of clusters. In some instances, a device comprises at least 10, 500, 1000, 2000, 3000, 4000, 5000, 6000, 7000, 8000, 9000, 10000, 11000, 12000, 13000, 14000, 15000, 20000, 30000, 40000, 50000 or more clusters. In some instances, a device comprises more than 2,000; 5,000; 10,000; 100,000; 200,000; 300,000; 400,000; 500,000; 600,000; 700,000; 800,000; 900,000; 1,000,000; 1,100,000; 1,200,000; 1,300,000; 1,400,000; 1,500,000; 1,600,000; 1,700,000; 1,800,000; 1,900,000; 2,000,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; or 10,000,000 or more distinct loci. In some instances, a device comprises about 10,000 distinct loci. The amount of loci within a single cluster is varied in different instances. In some instances, each cluster includes 1, 2, 3, 4, 5, 6, 7, 8, 9, 10, 20, 30, 40, 50, 60, 70, 80, 90, 100, 120, 130, 150, 200, 300, 400, 500 or more loci. In some instances, each cluster includes about 50-500 loci. In some instances, each cluster includes about 100-200 loci. In some instances, each cluster includes about 100-150 loci. In some instances, each cluster includes about 109, 121, 130 or 137 loci. In some instances, each cluster includes about 19, 20, 61, 64 or more loci.

The number of distinct oligonucleic acids synthesized on a device may be dependent on the number of distinct loci available in the substrate. In some instances, the density of loci within a cluster of a device is at least or about 1 locus per mm², 10 loci per mm², 25 loci per mm², 50 loci per mm², 65 loci per mm², 75 loci per mm², 100 loci per mm², 130 loci per mm², 150 loci per mm², 175 loci per mm², 200 loci per mm², 300 loci per mm², 400 loci per mm², 500 loci per mm², 1,000 loci per mm² or more. In some instances, a device comprises from about 10 loci per mm² to about 500 mm², from about 25 loci per mm² to about 400 mm², from about 50 loci per mm² to about 500 mm², from about 100 loci per mm² to about 500 mm², from about 150 loci per mm² to about 500 mm², from about 10 loci per mm² to about 250 mm², from about 50 loci per mm² to about 250 mm², from about 10 loci per mm² to about 200 mm², or from about 50 loci per mm² to about 200 mm². In some instances, the distance from the centers of two adjacent loci within a cluster is from about 10 um to about 500 um, from about 10 um to about 200 um, or from about 10 urn to about 100 um. In some instances, the distance from two centers of adjacent loci is greater than about 10 um, 20 urn, 30 um, 40 urn, 50 um, 60 um, 70 urn, 80 um, 90 um or 100 um. In some instances, the distance from the centers of two adjacent loci is less than about 200 um, 150 um, 100 um, 80 um, 70 um, 60 um, 50 um, 40 um, 30 um, 20 um or 10 um. In some instances, each locus has a width of about 0.5 um, 1 um, 2 urn, 3 um, 4 um, 5 um, 6 um, 7 um, 8 urn, 9 um, 10 um, 20 um, 30 um, 40 um, 50 um, 60 um, 70 urn, 80 um, 90 urn or 100 um. In some instances, the each locus is has a width of about 0.5 um to 100 um, about 0.5 um to 50 um, about 10 urn to 75 urn, or about 0.5 urn to 50 um.

In some instances, the density of clusters within a device is at least or about 1 cluster per 100 mm², 1 cluster per 10 mm², 1 cluster per 5 mm², 1 cluster per 4 mm², 1 cluster per 3 mm², 1 cluster per 2 mm², 1 cluster per 1 mm², 2 clusters per 1 mm², 3 clusters per 1 mm², 4 clusters per 1 mm², 5 clusters per 1 mm², 10 clusters per 1 mm², 50 clusters per 1 mm² or more. In some instances, a device comprises from about 1 cluster per 10 mm² to about 10 clusters per 1 mm². In some instances, the distance from the centers of two adjacent clusters is less than about 50 um, 100 um, 200 um, 500 urn, 1000 urn, or 2000 um or 5000 um. In some instances, the distance from the centers of two adjacent clusters is from about 50 um and about 100 um, from about 50 urn and about 200 um, from about 50 um and about 300 urn, from about 50 um and about 500 um, and from about 100 um to about 2000 um. In some instances, the distance from the centers of two adjacent clusters is from about 0.05 mm to about 50 mm, from about 0.05 mm to about 10 mm, from about 0.05 mm and about 5 mm, from about 0.05 mm and about 4 mm, from about 0.05 mm and about 3 mm, from about 0.05 mm and about 2 mm, from about 0.1 mm and 10 mm, from about 0.2 mm and 10 mm, from about 0.3 mm and about 10 mm, from about 0.4 mm and about 10 mm, from about 0.5 mm and 10 mm, from about 0.5 mm and about 5 mm, or from about 0.5 mm and about 2 mm. In some instances, each cluster has a diameter or width along one dimension of about 0.5 to 2 mm, about 0.5 to 1 mm, or about 1 to 2 mm. In some instances, each cluster has a diameter or width along one dimension of about 0.5, 0.6, 0.7, 0.8, 0.9, 1, 1.1, 1.2, 1.3, 1.4, 1.5, 1.6, 1.7, 1.8, 1.9 or 2 mm. In some instances, each cluster has an interior diameter or width along one dimension of about 0.5, 0.6, 0.7, 0.8, 0.9, 1, 1.1, 1.15, 1.2, 1.3, 1.4, 1.5, 1.6, 1.7, 1.8, 1.9 or 2 mm.

A device may be about the size of a standard 96 well plate, for example from about 100 and 200 mm by from about 50 and 150 mm. In some instances, a device has a diameter less than or equal to about 1000 mm, 500 mm, 450 mm, 400 mm, 300 mm, 250 nm, 200 mm, 150 mm, 100 mm or 50 mm. In some instances, the diameter of a device is from about 25 mm and 1000 mm, from about 25 mm and about 800 mm, from about 25 mm and about 600 mm, from about 25 mm and about 500 mm, from about 25 mm and about 400 mm, from about 25 mm and about 300 mm, or from about 25 mm and about 200. Non-limiting examples of device size include about 300 mm, 200 mm, 150 mm, 130 mm, 100 mm, 76 mm, 51 mm and 25 mm. In some instances, a device has a planar surface area of at least about 100 mm²; 200 mm²; 500 mm²; 1,000 mm²; 2,000 mm²; 5,000 mm²; 10,000 mm²; 12,000 mm²; 15,000 mm²; 20,000 mm²; 30,000 mm²; 40,000 mm²; 50,000 mm² or more. In some instances, the thickness of a device is from about 50 mm and about 2000 mm, from about 50 mm and about 1000 mm, from about 100 mm and about 1000 mm, from about 200 mm and about 1000 mm, or from about 250 mm and about 1000 mm. Non-limiting examples of device thickness include 275 mm, 375 mm, 525 mm, 625 mm, 675 mm, 725 mm, 775 mm and 925 mm. In some instances, the thickness of a device varies with diameter and depends on the composition of the substrate. For example, a device comprising materials other than silicon has a different thickness than a silicon device of the same diameter. Device thickness may be determined by the mechanical strength of the material used and the device must be thick enough to support its own weight without cracking during handling. In some instances, a structure comprises a plurality of devices described herein.

Surface Materials

Substrates, devices and reactors provided herein are fabricated from any variety of materials suitable for the methods and compositions described herein. In certain instances, device materials are fabricated to exhibit a low level of nucleotide binding. In some instances, device materials are modified to generate distinct surfaces that exhibit a high level of nucleotide binding. In some instances, device materials are transparent to visible and/or UV light. In some instances, device materials are sufficiently conductive, e.g., are able to form uniform electric fields across all or a portion of a substrate. In some instances, conductive materials are connected to an electric ground. In some instances, the device is heat conductive or insulated. In some instances, the materials are chemical resistant and heat resistant to support chemical or biochemical reactions, for example oligonucleic acid synthesis reaction processes. In some instances, a device comprises flexible materials. Flexible materials include, without limitation, modified nylon, unmodified nylon, nitrocellulose, polypropylene, and the like. In some instances, a device comprises rigid materials. Rigid materials include, without limitation, glass, fuse silica, silicon, silicon dioxide, silicon nitride, plastics (for example, polytetraflouroethylene, polypropylene, polystyrene, polycarbonate, and blends thereof and the like), and metals (for example, gold, platinum, and the like). In some instances, a device is fabricated from a material comprising silicon, polystyrene, agarose, dextran, cellulosic polymers, polyacrylamides, polydimethylsiloxane (PDMS), glass, or any combination thereof. In some instances, a device is manufactured with a combination of materials listed herein or any other suitable material known in the art.

Surface Architecture

Provided herein are devices comprising raised and/or lowered features. One benefit of having such features is an increase in surface area to support oligonucleic acid synthesis. In some instances, a device having raised and/or lowered features is referred to as a three-dimensional substrate. In some instances, a three-dimensional device comprises one or more channels. In some instances, one or more loci comprise a channel. In some instances, the channels are accessible to reagent deposition via a deposition device such as an oligonucleic acid synthesizer. In some instances, reagents and/or fluids collect in a larger well in fluid communication one or more channels. For example, a device comprises a plurality of channels corresponding to a plurality of loci with a cluster, and the plurality of channels are in fluid communication with one well of the cluster. In some methods, a library of oligonucleic acids is synthesized in a plurality of loci of a cluster.

In some instances, the structure is configured to allow for controlled flow and mass transfer paths for oligonucleic acid synthesis on a surface. In some instances, the configuration of a device allows for the controlled and even distribution of mass transfer paths, chemical exposure times, and/or wash efficacy during oligonucleic acid synthesis. In some instances, the configuration of a device allows for increased sweep efficiency, for example by providing sufficient volume for a growing an oligonucleic acid such that the excluded volume by the growing oligonucleic acid does not take up more than 50, 45, 40, 35, 30, 25, 20, 15, 14, 13, 12, 11, 10, 9, 8, 7, 6, 5, 4, 3, 2, 1%, or less of the initially available volume that is available or suitable for growing the oligonucleic acid. In some instances, a three-dimensional structure allows for managed flow of fluid to allow for the rapid exchange of chemical exposure.

Provided herein are methods to synthesize an amount of DNA of 1 fM, 5 fM, 10 fM, 25 fM, 50 fM, 75 fM, 100 fM, 200 fM, 300 fM, 400 fM, 500 fM, 600 fM, 700 fM, 800 fM, 900 fM, 1 pM, 5 pM, 10 pM, 25 pM, 50 pM, 75 pM, 100 pM, 200 pM, 300 pM, 400 pM, 500 pM, 600 pM, 700 pM, 800 pM, 900 pM, or more. In some instances, an oligonucleotide library may span the length of about 1%, 2%, 3%, 4%, 5%, 10%, 159%, 20%, 30%, 40%, 50%, 60%, 70%, 80%, 90%, 95%, or 100% of a gene. A gene may be varied up to about 1%, 2%, 3%, 4%, 5%, 10%, 15%, 20%, 30%, 40%, 509%, 60%, 70%, 80%, 85%, 90%, 95%, or 100%.

Non-identical oligonucleic acids may collectively encode a sequence for at least 1%, 2%, 3%, 4%, 5%, 10%, 15%, 20%, 30%, 40%, 50%, 60%, 70%, 80%, 85%, 90%, 95%, or 100% of a gene. In some instances, an oligonucleic acid may encode a sequence of 50%, 60%, 70%, 80%, 85%, 90%, 95%, or more of a gene. In some instances, an oligonucleic acid may encode a sequence of 80%, 85%, 90%, 95%, or more of a gene.

In some instances, segregation is achieved by physical structure. In some instances, segregation is achieved by differential functionalization of the surface generating active and passive regions for oligonucleic acid synthesis. Differential functionalization is also be achieved by alternating the hydrophobicity across the device surface, thereby creating water contact angle effects that cause beading or wetting of the deposited reagents. Employing larger structures can decrease splashing and cross-contamination of distinct oligonucleic acid synthesis locations with reagents of the neighboring spots. In some instances, a device, such as an oligonucleic acid synthesizer, is used to deposit reagents to distinct oligonucleic acid synthesis locations. Substrates having three-dimensional features are configured in a manner that allows for the synthesis of a large number of oligonucleic acids (e.g., more than about 10,000) with a low error rate (e.g., less than about 1:500, 1:1000, 1:1500, 1:2,000; 1:3,000; 1:5,000; or 1:10,000). In some instances, a device comprises features with a density of about or greater than about 1, 5, 10, 20, 30, 40, 50, 60, 70, 80, 100, 110, 120, 130, 140, 150, 160, 170, 180, 190, 200, 300, 400 or 500 features per mm².

A well of a device may have the same or different width, height, and/or volume as another well of the substrate. A channel of a device may have the same or different width, height, and/or volume as another channel of the substrate. In some instances, the width of a cluster is from about 0.05 mm to about 50 mm, from about 0.05 mm to about 10 mm, from about 0.05 mm and about 5 mm, from about 0.05 mm and about 4 mm, from about 0.05 mm and about 3 mm, from about 0.05 mm and about 2 mm, from about 0.05 mm and about 1 mm, from about 0.05 mm and about 0.5 mm, from about 0.05 mm and about 0.1 mm, from about 0.1 mm and 10 mm, from about 0.2 mm and 10 mm, from about 0.3 mm and about 10 mm, from about 0.4 mm and about 10 mm, from about 0.5 mm and 10 mm, from about 0.5 mm and about 5 mm, or from about 0.5 mm and about 2 mm. In some instances, the width of a well comprising a cluster is from about 0.05 mm to about 50 mm, from about 0.05 mm to about 10 mm, from about 0.05 mm and about 5 mm, from about 0.05 mm and about 4 mm, from about 0.05 mm and about 3 mm, from about 0.05 mm and about 2 mm, from about 0.05 mm and about 1 mm, from about 0.05 mm and about 0.5 mm, from about 0.05 mm and about 0.1 mm, from about 0.1 mm and 10 mm, from about 0.2 mm and 10 mm, from about 0.3 mm and about 10 mm, from about 0.4 mm and about 10 mm, from about 0.5 mm and 10 mm, from about 0.5 mm and about 5 mm, or from about 0.5 mm and about 2 mm. In some instances, the width of a cluster is less than or about 5 mm, 4 mm, 3 mm, 2 mm, 1 mm, 0.5 mm, 0.1 mm, 0.09 mm, 0.08 mm, 0.07 mm, 0.06 mm or 0.05 mm. In some instances, the width of a cluster is from about 1.0 and 1.3 mm. In some instances, the width of a cluster is about 1.150 mm. In some instances, the width of a well is less than or about 5 mm, 4 mm, 3 mm, 2 mm, 1 mm, 0.5 mm, 0.1 mm, 0.09 mm, 0.08 mm, 0.07 mm, 0.06 mm or 0.05 mm. In some instances, the width of a well is from about 1.0 and 1.3 mm. In some instances, the width of a well is about 1.150 mm. In some instances, the width of a cluster is about 0.08 mm. In some instances, the width of a well is about 0.08 mm. The width of a duster may refer to clusters within a two-dimensional or three-dimensional substrate.

In some instances, the height of a well is from about 20 um to about 1000 um, from about 50 um to about 1000 um, from about 100 um to about 1000 um, from about 200 um to about 1000 um, from about 300 um to about 1000 um, from about 400 um to about 1000 um, or from about 500 um to about 1000 um. In some instances, the height of a well is less than about 1000 um, less than about 900 um, less than about 800 um, less than about 700 um, or less than about 600 um.

In some instances, a device comprises a plurality of channels corresponding to a plurality of loci within a duster, wherein the height or depth of a channel is from about 5 um to about 500 um, from about 5 um to about 400 um, from about 5 um to about 300 um, from about 5 um to about 200 um, from about 5 um to about 100 um, from about 5 um to about 50 um, or from about 10 um to about 50 um. In some instances, the height of a channel is less than 100 um, less than 80 um, less than 60 um, less than 40 um or less than 20 um.

In some instances, the diameter of a channel, locus (e.g., in a substantially planar substrate) or both channel and locus (e.g., in a three-dimensional device wherein a locus corresponds to a channel) is from about 1 um to about 1000 um, from about 1 um to about 500 um, from about 1 um to about 200 um, from about 1 um to about 100 um, from about 5 um to about 100 um, or from about 10 um to about 100 um, for example, about 90 um, 80 um, 70 um, 60 um, 50 um, 40 um, 30 um, 20 um or 10 um. In some instances, the diameter of a channel, locus, or both channel and locus is less than about 100 um, 90 um, 80 um, 70 um, 60 um, 50 um, 40 um, 30 um, 20 um or 10 um. In some instances, the distance from the center of two adjacent channels, loci, or channels and loci is from about 1 um to about 500 um, from about 1 um to about 200 um, from about 1 um to about 100 um, from about 5 urn to about 200 um, from about 5 urn to about 100 um, from about 5 um to about 50 um, or from about 5 urn to about 30 um, for example, about 20 um.

Surface Modifications

In various instances, surface modifications are employed for the chemical and/or physical alteration of a surface by an additive or subtractive process to change one or more chemical and/or physical properties of a device surface or a selected site or region of a device surface. For example, surface modifications include, without limitation, (1) changing the wetting properties of a surface, (2) functionalizing a surface, i.e., providing, modifying or substituting surface functional groups, (3) defunctionalizing a surface, i.e., removing surface functional groups, (4) otherwise altering the chemical composition of a surface, e.g., through etching, (5) increasing or decreasing surface roughness, (6) providing a coating on a surface, e.g., a coating that exhibits wetting properties that are different from the wetting properties of the surface, and/or (7) depositing particulates on a surface.

In some instances, the addition of a chemical layer on top of a surface (referred to as adhesion promoter) facilitates structured patterning of loci on a surface of a substrate. Exemplary surfaces for application of adhesion promotion include, without limitation, glass, silicon, silicon dioxide and silicon nitride. In some instances, the adhesion promoter is a chemical with a high surface energy. In some instances, a second chemical layer is deposited on a surface of a substrate. In some instances, the second chemical layer has a low surface energy. In some instances, surface energy of a chemical layer coated on a surface supports localization of droplets on the surface. Depending on the patterning arrangement selected, the proximity of loci and/or area of fluid contact at the loci are alterable.

In some instances, a device surface, or resolved loci, onto which nucleic acids or other moieties are deposited, e.g., for oligonucleic acid synthesis, are smooth or substantially planar (e.g., two-dimensional) or have irregularities, such as raised or lowered features (e.g., three-dimensional features). In some instances, a device surface is modified with one or more different layers of compounds. Such modification layers of interest include, without limitation, inorganic and organic layers such as metals, metal oxides, polymers, small organic molecules and the like. Non-limiting polymeric layers include peptides, proteins, nucleic acids or mimetics thereof (e.g., peptide nucleic acids and the like), polysaccharides, phospholipids, polyurethanes, polyesters, polycarbonates, polyureas, polyamides, polyetheyleneamines, polyarylene sulfides, polysiloxanes, polyimides, polyacetates, and any other suitable compounds described herein or otherwise known in the art. In some instances, polymers are heteropolymeric. In some instances, polymers are homopolymeric. In some instances, polymers comprise functional moieties or are conjugated.

In some instances, resolved loci of a device are functionalized with one or more moieties that increase and/or decrease surface energy. In some instances, a moiety is chemically inert. In some instances, a moiety is configured to support a desired chemical reaction, for example, one or more processes in an oligonucleic acid synthesis reaction. The surface energy, or hydrophobicity, of a surface is a factor for determining the affinity of a nucleotide to attach onto the surface. In some instances, a method for device functionalization may comprise: (a) providing a device having a surface that comprises silicon dioxide; and (b) silanizing the surface using, a suitable silanizing agent described herein or otherwise known in the art, for example, an organofunctional alkoxysilane molecule.

In some instances, the organofunctional alkoxysilane molecule comprises dimethylchloro-octodecyl-silane, methyldichloro-octodecyl-silane, trichloro-octodecyl-silane, trimethyl-octodecyl-silane, triethyl-octodecyl-silane, or any combination thereof. In some instances, a device surface comprises functionalized with polyethylene/polypropylene (functionalized by gamma irradiation or chromic acid oxidation, and reduction to hydroxyalkyl surface), highly crosslinked polystyrene-divinylbenzene (derivatized by chloromethylation, and aminated to benzylamine functional surface), nylon (the terminal aminohexyl groups are directly reactive), or etched with reduced polytetrafluoroethylene. Other methods and functionalizing agents are described in U.S. Pat. No. 5,474,796, which is herein incorporated by reference in its entirety.

In some instances, a device surface is functionalized by contact with a derivatizing composition that contains a mixture of silanes, under reaction conditions effective to couple the silanes to the device surface, typically via reactive hydrophilic moieties present on the device surface. Silanization generally covers a surface through self-assembly with organofunctional alkoxysilane molecules.

A variety of siloxane functionalizing reagents can further be used as currently known in the art, e.g., for lowering or increasing surface energy. The organofunctional alkoxysilanes can be classified according to their organic functions.

Provided herein are devices that may contain patterning of agents capable of coupling to a nucleoside. In some instances, a device may be coated with an active agent. In some instances, a device may be coated with a passive agent. Exemplary active agents for inclusion in coating materials described herein includes, without limitation, N-(3-triethoxysilylpropyl)-4-hydroxybutyramide (HAPS), 11-acetoxyundecyltriethoxysilane, n-decyltriethoxysilane, (3-aminopropyl)trimethoxysilane, (3-aminopropyl)triethoxysilane, 3-glycidoxypropyltrimethoxysilane (GOPS), 3-iodo-propyltrimethoxysilane, butyl-aldehydr-trimethoxysilane, dimeric secondary aminoalkyl siloxanes, (3-aminopropyl)-diethoxy-methylsilane, (3-aminopropyl)-dimethyl-ethoxysilane, and (3-aminopropyl)-trimethoxysilane, (3-glycidoxypropyl)-dimethyl-ethoxysilane, glycidoxy-trimethoxysilane, (3-mercaptopropyl)-trimethoxysilane, 3-4 epoxycyclohexyl-ethyltrimethoxysilane, and (3-mercaptopropyl)-methyl-dimethoxysilane, allyl trichlorochlorosilane, 7-oct-1-enyl trichlorochlorosilane, or bis (3-trimethoxysilylpropyl) amine.

Exemplary passive agents for inclusion in a coating material described herein includes, without limitation, perfluorooctyltrichlorosilane; tridecafluoro-1,1,2,2-tetrahydrooctyl)trichlorosilane; 1H, 1H, 2H, 2H-fluorooctyltriethoxysilane (FOS); trichloro(1H, 1H, 2H, 2H-perfluorooctyl)silane; tert-butyl-[5-fluoro-4-(4,4,5,5-tetramethyl-1,3,2-dioxaborolan-2-yl)indol-1-yl]-dimethyl-silane; CYTOP™; Fluorinert™; perfluoroctyltrichlorosilane (PFOTCS); perfluorooctyldimethylchlorosilane (PFODCS); perfluorodecyltriethoxysilane (PFDTES); pentafluorophenyl-dimethylpropylchloro-silane (PFPTES); perfluorooctyltriethoxysilane; perfluorooctyltrimethoxysilane; octylchlorosilane; dimethylchloro-octodecyl-silane; methyldichloro-octodecyl-silane; trichloro-octodecyl-silane; trimethyl-octodecyl-silane; triethyl-octodecyl-silane; or octadecyltrichlorosilane.

In some instances, a functionalization agent comprises a hydrocarbon silane such as octadecyltrichlorosilane. In some instances, the functionalizing agent comprises 11-acetoxyundecyltriethoxysilane, n-decyltriethoxysilane, (3-aminopropyl)trimethoxysilane, (3-aminopropyl)triethoxysilane, glycidyloxypropyl/trimethoxysilane and N-(3-triethoxysilylpropyl)-4-hydroxybutyramide.

Oligonucleotide Synthesis

Methods of the current disclosure for oligonucleic acid synthesis may include processes involving phosphoramidite chemistry. In some instances, oligonucleic acid synthesis comprises coupling a base with phosphoramidite. Oligonucleic acid synthesis may comprise coupling a base by deposition of phosphoramidite under coupling conditions, wherein the same base is optionally deposited with phosphoramidite more than once, i.e., double coupling. Oligonucleic acid synthesis may comprise capping of unreacted sites. In some instances, capping is optional. Oligonucleic acid synthesis may also comprise oxidation or an oxidation step or oxidation steps. Oligonucleic acid synthesis may comprise deblocking, detritylation, and sulfurization. In some instances, oligonucleic acid synthesis comprises either oxidation or sulfurization. In some instances, between one or each step during an oligonucleic acid synthesis reaction, the device is washed, for example, using tetrazole or acetonitrile. Time frames for any one step in a phosphoramidite synthesis method may be less than about 2 min, 1 min, 50 sec, 40 sec, 30 sec, 20 sec and 10 sec.

Oligonucleic acid synthesis using a phosphoramidite method may comprise a subsequent addition of a phosphoramidite building block (e.g., nucleoside phosphoramidite) to a growing oligonucleic acid chain for the formation of a phosphite triester linkage. Phosphoramidite oligonucleic acid synthesis proceeds in the 3′ to 5′ direction. Phosphoramidite oligonucleic acid synthesis allows for the controlled addition of one nucleotide to a growing nucleic acid chain per synthesis cycle. In some instances, each synthesis cycle comprises a coupling step. Phosphoramidite coupling involves the formation of a phosphite triester linkage between an activated nucleoside phosphoramidite and a nucleoside bound to the substrate, for example, via a linker. In some instances, the nucleoside phosphoramidite is provided to the device activated. In some instances, the nucleoside phosphoramidite is provided to the device with an activator. In some instances, nucleoside phosphoramidites are provided to the device in a 1.5, 2, 3, 4, 5, 6, 7, 8, 9, 10, 11, 12, 13, 14, 15, 16, 17, 18, 19, 20, 25, 30, 35, 40, 50, 60, 70, 80, 90, 100-fold excess or more over the substrate-bound nucleosides. In some instances, the addition of nucleoside phosphoramidite is performed in an anhydrous environment, for example, in anhydrous acetonitrile. Following addition of a nucleoside phosphoramidite, the device is optionally washed. In some instances, the coupling step is repeated one or more additional times, optionally with a wash step between nucleoside phosphoramidite additions to the substrate. In some instances, an oligonucleic acid synthesis method used herein comprises 1, 2, 3 or more sequential coupling steps. Prior to coupling, in many cases, the nucleoside bound to the device is de-protected by removal of a protecting group, where the protecting group functions to prevent polymerization. A common protecting group is 4,4′-dimethoxytrityl (DMT).

Following coupling, phosphoramidite oligonucleic acid synthesis methods optionally comprise a capping step. In a capping step, the growing oligonucleic acid is treated with a capping agent. A capping step is useful to block unreacted substrate-bound 5′-OH groups after coupling from further chain elongation, preventing the formation of oligonucleic acids with internal base deletions. Further, phosphoramidites activated with 1H-tetrazole may react, to a small extent, with the O6 position of guanosine. Without being bound by theory, upon oxidation with I₂/water, this side product, possibly via O6-N7 migration, may undergo depurination. The apurinic sites may end up being cleaved in the course of the final deprotection of the oligonucleotide thus reducing the yield of the full-length product. The O6 modifications may be removed by treatment with the capping reagent prior to oxidation with I₂/water. In some instances, inclusion of a capping step during oligonucleic acid synthesis decreases the error rate as compared to synthesis without capping. As an example, the capping step comprises treating the substrate-bound oligonucleic acid with a mixture of acetic anhydride and 1-methylimidazole. Following a capping step, the device is optionally washed.

In some instances, following addition of a nucleoside phosphoramidite, and optionally after capping and one or more wash steps, the device bound growing nucleic acid is oxidized. The oxidation step comprises the phosphite triester is oxidized into a tetracoordinated phosphate triester, a protected precursor of the naturally occurring phosphate diester internucleoside linkage. In some instances, oxidation of the growing oligonucleic acid is achieved by treatment with iodine and water, optionally in the presence of a weak base (e.g., pyridine, lutidine, collidine). Oxidation may be carried out under anhydrous conditions using, e.g. tert-Butyl hydroperoxide or (1S)-(+)-(10-camphorsulfonyl)-oxaziridine (CSO). In some methods, a capping step is performed following oxidation. A second capping step allows for device drying, as residual water from oxidation that may persist can inhibit subsequent coupling. Following oxidation, the device and growing oligonucleic acid is optionally washed. In some instances, the step of oxidation is substituted with a sulfurization step to obtain oligonucleotide phosphorothioates, wherein any capping steps can be performed after the sulfurization. Many reagents are capable of the efficient sulfur transfer, including but not limited to 3-(Dimethylaminomethylidene)amino)-3H-1,2,4-dithiazole-3-thione, DDTT, 3H-1,2-benzodithiol-3-one 1,1-dioxide, also known as Beaucage reagent, and N,N,N′N′-Tetraethylthiuram disulfide (TETD).

In order for a subsequent cycle of nucleoside incorporation to occur through coupling, the protected 5′ end of the device bound growing oligonucleic acid is removed so that the primary hydroxyl group is reactive with a next nucleoside phosphoramidite. In some instances, the protecting group is DMT and deblocking occurs with trichloroacetic acid in dichloromethane. Conducting detritylation for an extended time or with stronger than recommended solutions of acids may lead to increased depurination of solid support-bound oligonucleotide and thus reduces the yield of the desired full-length product. Methods and compositions of the disclosure described herein provide for controlled deblocking conditions limiting undesired depurination reactions. In some instances, the device bound oligonucleic acid is washed after deblocking. In some instances, efficient washing after deblocking contributes to synthesized oligonucleic acids having a low error rate.

Methods for the synthesis of oligonucleic acids typically involve an iterating sequence of the following steps: application of a protected monomer to an actively functionalized surface (e.g., locus) to link with either the activated surface, a linker or with a previously deprotected monomer; deprotection of the applied monomer so that it is reactive with a subsequently applied protected monomer; and application of another protected monomer for linking. One or more intermediate steps include oxidation or sulfurization. In some instances, one or more wash steps precede or follow one or all of the steps.

Methods for phosphoramidite-based oligonucleic acid synthesis comprise a series of chemical steps. In some instances, one or more steps of a synthesis method involve reagent cycling, where one or more steps of the method comprise application to the device of a reagent useful for the step. For example, reagents are cycled by a series of liquid deposition and vacuum drying steps. For substrates comprising three-dimensional features such as wells, microwells, channels and the like, reagents are optionally passed through one or more regions of the device via the wells and/or channels.

Methods and systems described herein relate to oligonucleotide synthesis devices for the synthesis of oligonucleotides. The synthesis may be in parallel. For example at least or about at least 2, 3, 4, 5, 6, 7, 8, 9, 10, 11, 12, 13, 14, 15, 16, 17, 18, 19, 20, 21, 22, 23, 24, 25, 30, 35, 40, 45, 50, 100, 150, 200, 250, 300, 350, 400, 450, 500, 550, 600, 650, 700, 750, 800, 850, 900, 1000, 10000, 50000, 75000, 100000 or more oligonucleotides can be synthesized in parallel. The total number oligonucleic acids that may be synthesized in parallel may be from 2-100000, 3-50000, 4-10000, 5-1000, 6-900, 7-850, 8-800, 9-750, 10-700, 11-650, 12-600, 13-550, 14-500, 15-450, 16-400, 17-350, 18-300, 19-250, 20-200, 21-150,22-100, 23-50, 24-45, 25-40, 30-35. Those of skill in the art appreciate that the total number of oligonucleotides synthesized in parallel may fall within any range bound by any of these values, for example 25-100. The total number of oligonucleotides synthesized in parallel may fall within any range defined by any of the values serving as endpoints of the range. Total molar mass of oligonucleotides synthesized within the device or the molar mass of each of the oligonucleotides may be at least or at least about 10, 20, 30, 40, 50, 100, 250, 500, 750, 1000, 2000, 3000, 4000, 5000, 6000, 7000, 8000, 9000, 10000, 25000, 50000, 75000, 100000 picomoles, or more. The length of each of the oligonucleotides or average length of the oligonucleotides within the device may be at least or about at least 10, 15, 20, 25, 30, 35, 40, 45, 50, 100, 150, 200, 300, 400, 500 nucleotides, or more. The length of each of the oligonucleotides or average length of the oligonucleotides within the device may be at most or about at most 500, 400, 300, 200, 150, 100, 50, 45, 35, 30, 25, 20, 19, 18, 17, 16, 15, 14, 13, 12, 11, 10 nucleotides, or less. The length of each of the oligonucleotides or average length of the oligonucleotides within the device may fall from 10-500, 9-400, 11-300, 12-200, 13-150, 14-100, 15-50, 16-45, 17-40, 18-35, 19-25. Those of skill in the art appreciate that the length of each of the oligonucleotides or average length of the oligonucleotides within the device may fall within any range bound by any of these values, for example 100-300. The length of each of the oligonucleotides or average length of the oligonucleotides within the device may fall within any range defined by any of the values serving as endpoints of the range.

Methods for oligonucleic acid synthesis on a surface provided herein allow for synthesis at a fast rate. As an example, at least 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 70, 80, 90, 100, 125, 150, 175, 200 nucleotides per hour, or more are synthesized. Nucleotides include adenine, guanine, thymine, cytosine, uridine building blocks, or analogs/modified versions thereof. In some instances, libraries of oligonucleic acids are synthesized in parallel on substrate. For example, a device comprising about or at least about 100; 1,000; 10,000; 30,000; 75,000; 100,000; 1,000,000; 2,000,000; 3,000,000; 4,000,000; or 5,000,000 resolved loci is able to support the synthesis of at least the same number of distinct oligonucleic acids, wherein oligonucleic acid encoding a distinct sequence is synthesized an a resolved locus. In some instances, a library of oligonucleic acids are synthesized on a device with low error rates described herein in less than about three months, two months, one month, three weeks, 15, 14, 13, 12, 11, 10, 9, 8, 7, 6, 5, 4, 3, 2 days, 24 hours or less. In some instances, larger nucleic acids assembled from an oligonucleic acid library synthesized with low error rate using the substrates and methods described herein are prepared in less than about three months, two months, one month, three weeks, 15, 14, 13, 12, 11, 10, 9, 8, 7, 6, 5, 4, 3, 2 days, 24 hours or less.

In some instances, methods described herein provide for generation of a library of oligonucleic acids comprising variant oligonucleic acids differing at a plurality of codon sites. In some instances, an oligonucleic acid may have 1 site, 2 sites, 3 sites, 4 sites, 5 sites, 6 sites, 7 sites, 8 sites, 9 sites, 10 sites, 11 sites, 12 sites, 13 sites, 14 sites, 15 sites, 16 sites, 17 sites 18 sites, 19 sites, 20 sites, 30 sites, 40 sites, 50 sites, or more of variant codon sites.

In some instances, the one or more sites of variant codon sites may be adjacent. In some instances, the one or more sites of variant codon sites may be not be adjacent and separated by 1, 2, 3, 4, 5, 6, 7, 8, 9, 10, or more codons.

In some instances, an oligonucleic acid may comprise multiple sites of variant codon sites, wherein all the variant codon sites are adjacent to one another, forming a stretch of variant codon sites. In some instances, an oligonucleic acid may comprise multiple sites of variant codon sites, wherein none the variant codon sites are adjacent to one another. In some instances, an oligonucleic acid may comprise multiple sites of variant codon sites, wherein some the variant codon sites are adjacent to one another, forming a stretch of variant codon sites, and some of the variant codon sites are not adjacent to one another.

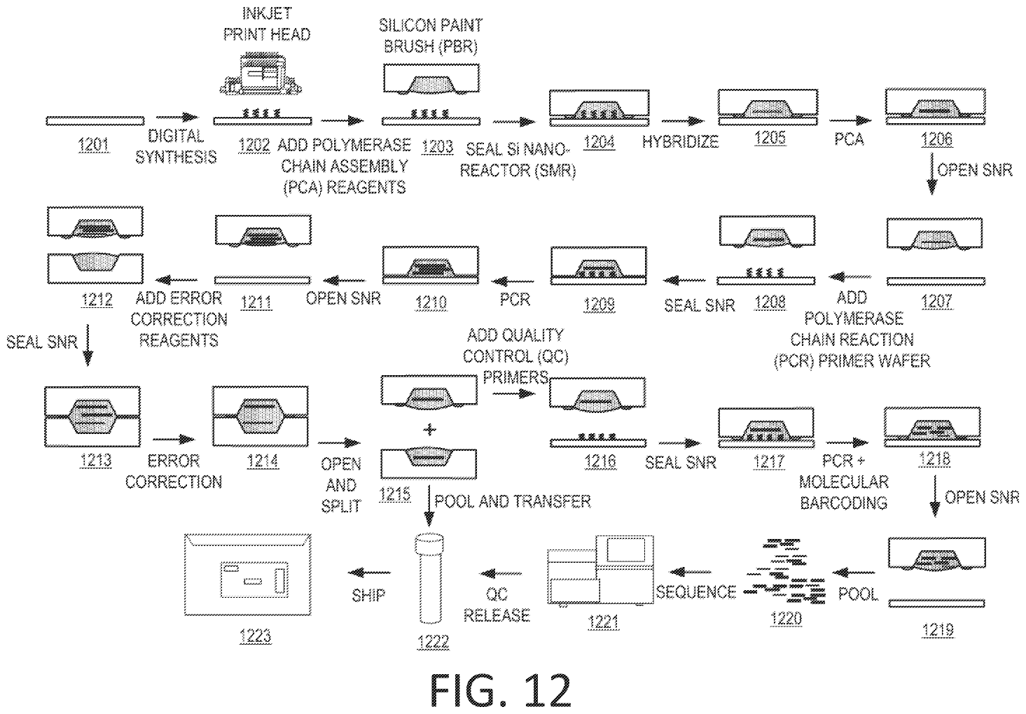

Referring to the Figures, FIG. 12 illustrates an exemplary process workflow for synthesis of nucleic acids (e.g., genes) from shorter oligonucleic acids. The workflow is divided generally into phases: (1) de novo synthesis of a single stranded oligonucleic acid library, (2) joining oligonucleic acids to form larger fragments, (3) error correction, (4) quality control, and (5) shipment. Prior to de novo synthesis, an intended nucleic acid sequence or group of nucleic acid sequences is preselected. For example, a group of genes is preselected for generation.

Once large oligonucleic acids for generation are selected, a predetermined library of oligonucleic acids is designed for de novo synthesis. Various suitable methods are known for generating high density oligonucleic acid arrays. In the workflow example, a device surface layer 1201 is provided. In the example, chemistry of the surface is altered in order to improve the oligonucleic acid synthesis process. Areas of low surface energy are generated to repel liquid while areas of high surface energy are generated to attract liquids. The surface itself may be in the form of a planar surface or contain variations in shape, such as protrusions or microwells which increase surface area. In the workflow example, high surface energy molecules selected serve a dual function of supporting DNA chemistry, as disclosed in International Patent Application Publication WO/2015/021080, which is herein incorporated by reference in its entirety.

In situ preparation of oligonucleic acid arrays is generated on a solid support and utilizes single nucleotide extension process to extend multiple oligomers in parallel. A material deposition device, such as an oligonucleic acid synthesizer, is designed to release reagents in a step wise fashion such that multiple oligonucleic acids extend, in parallel, one residue at a time to generate oligomers with a predetermined nucleic acid sequence 1202. In some instances, oligonucleic acids are cleaved from the surface at this stage. Cleavage includes gas cleavage, e.g., with ammonia or methylamine.

The generated oligonucleic acid libraries are placed in a reaction chamber. In this exemplary workflow, the reaction chamber (also referred to as “nanoreactor”) is a silicon coated well, containing PCR reagents and lowered onto the oligonucleic acid library 1203. Prior to or after the sealing 1204 of the oligonucleic acids, a reagent is added to release the oligonucleic acids from the substrate. In the exemplary workflow, the oligonucleic acids are released subsequent to sealing of the nanoreactor 1205. Once released, fragments of single stranded oligonucleic acids hybridize in order to span an entire long range sequence of DNA. Partial hybridization 1205 is possible because each synthesized oligonucleic acid is designed to have a small portion overlapping with at least one other oligonucleic acid in the pool.

After hybridization, a PCA reaction is commenced. During the polymerase cycles, the oligonucleic acids anneal to complementary fragments and gaps are filled in by a polymerase. Each cycle increases the length of various fragments randomly depending on which oligonucleic acids find each other. Complementarity amongst the fragments allows for forming a complete large span of double stranded DNA 1206.

After PCA is complete, the nanoreactor is separated from the device 1207 and positioned for interaction with a device having primers for PCR 1208. After sealing, the nanoreactor is subject to PCR 1209 and the larger nucleic acids are amplified. After PCR 1210, the nanochamber is opened 1211, error correction reagents are added 1212, the chamber is sealed 1213 and an error correction reaction occurs to remove mismatched base pairs and/or strands with poor complementarity from the double stranded PCR amplification products 1214. The nanoreactor is opened and separated 1215. Error corrected product is next subject to additional processing steps, such as PCR and molecular bar coding, and then packaged 1222 for shipment 1223.

In some instances, quality control measures are taken. After error correction, quality control steps include for example interaction with a wafer having sequencing primers for amplification of the error corrected product 1216, sealing the wafer to a chamber containing error corrected amplification product 1217, and performing an additional round of amplification 1218. The nanoreactor is opened 1219 and the products are pooled 1220 and sequenced 1221. After an acceptable quality control determination is made, the packaged product 1222 is approved for shipment 1223.

In some instances, a nucleic acid generate by a workflow such as that in FIG. 12 is subject to mutagenesis using overlapping primers disclosed herein. In some instances, a library of primers are generated by in situ preparation on a solid support and utilize single nucleotide extension process to extend multiple oligomers in parallel. A deposition device, such as an oligonucleic acid synthesizer, is designed to release reagents in a step wise fashion such that multiple oligonucleic acids extend, in parallel, one residue at a time to generate oligomers with a predetermined nucleic acid sequence 1202.

Computer Systems

Any of the systems described herein, may be operably linked to a computer and may be automated through a computer either locally or remotely. In various instances, the methods and systems of the disclosure may further comprise software programs on computer systems and use thereof. Accordingly, computerized control for the synchronization of the dispense/vacuum/refill functions such as orchestrating and synchronizing the material deposition device movement, dispense action and vacuum actuation are within the bounds of the disclosure. The computer systems may be programmed to interface between the user specified base sequence and the position of a material deposition device to deliver the correct reagents to specified regions of the substrate.

The computer system 1300 illustrated in FIG. 13 may be understood as a logical apparatus that can read instructions from media 1311 and/or a network port 1305, which can optionally be connected to server 1309 having fixed media 1312. The system, such as shown in FIG. 13 can include a CPU 1301, disk drives 1303, optional input devices such as keyboard 1315 and/or mouse 1316 and optional monitor 1307. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 1322 as illustrated in FIG. 13.

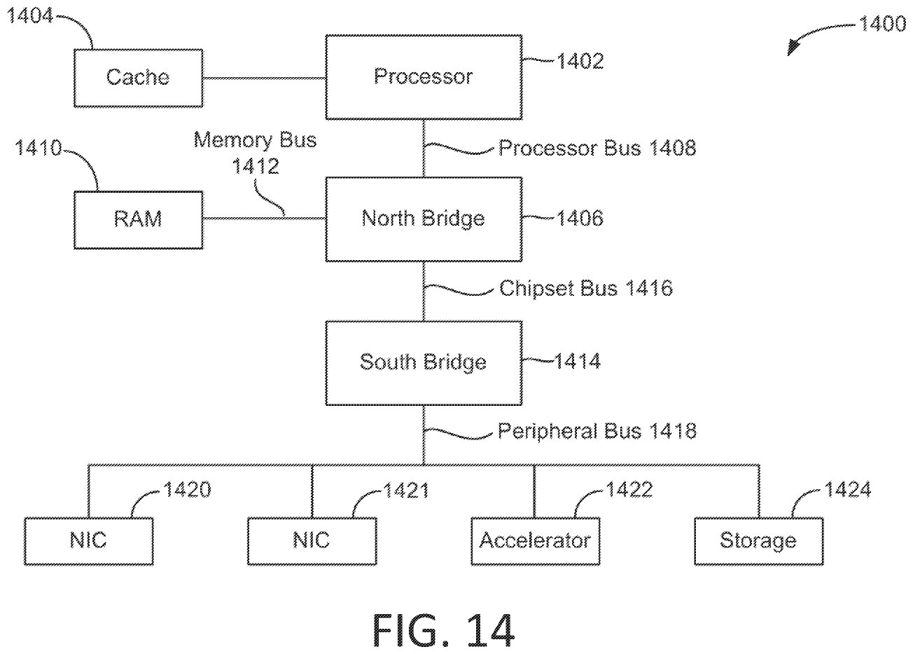

FIG. 14 is a block diagram illustrating a first example architecture of a computer system 1400 that can be used in connection with example instances of the present disclosure. As depicted in FIG. 14, the example computer system can include a processor 1402 for processing instructions. Non-limiting examples of processors include: Intel Xeon™ processor, AMD Opteron™ processor, Samsung 32-bit RISC ARM 1176JZ(F)-S v1.0™ processor, ARM Cortex-A8 Samsung S5PC100™ processor, ARM Cortex-A8 Apple A4™ processor, Marvell PXA 930™ processor, or a functionally-equivalent processor. Multiple threads of execution can be used for parallel processing. In some instances, multiple processors or processors with multiple cores can also be used, whether in a single computer system, in a cluster, or distributed across systems over a network comprising a plurality of computers, cell phones, and/or personal data assistant devices.

As illustrated in FIG. 14, a high speed cache 1404 can be connected to, or incorporated in, the processor 1402 to provide a high speed memory for instructions or data that have been recently, or are frequently, used by processor 1402. The processor 1402 is connected to a north bridge 1406 by a processor bus 1408. The north bridge 1406 is connected to random access memory (RAM) 1410 by a memory bus 1412 and manages access to the RAM 1410 by the processor 1402. The north bridge 1406 is also connected to a south bridge 1414 by a chipset bus 1416. The south bridge 1414 is, in turn, connected to a peripheral bus 1418. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 1418. In some alternative architectures, the functionality of the north bridge can be incorporated into the processor instead of using a separate north bridge chip. In some instances, system 1400 can include an accelerator card 1422 attached to the peripheral bus 1418. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 1424 and can be loaded into RAM 1410 and/or cache 1404 for use by the processor. The system 1400 includes an operating system for managing system resources; non-limiting examples of operating systems include: Linux, Windows™, MACOS™, BlackBerry OS™, iOS™, and other functionally-equivalent operating systems, as well as application software running on top of the operating system for managing data storage and optimization in accordance with example instances of the present disclosure. In this example, system 1400 also includes network interface cards (NICs) 1420 and 1421 connected to the peripheral bus for providing network interfaces to external storage, such as Network Attached Storage (NAS) and other computer systems that can be used for distributed parallel processing.

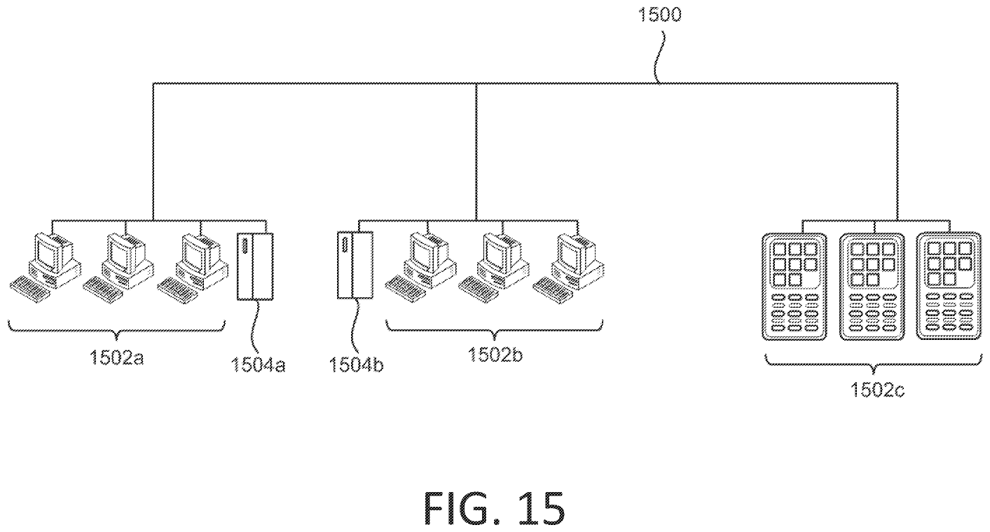

FIG. 15 is a diagram showing a network 1500 with a plurality of computer systems 1502a, and 1502b, a plurality of cell phones and personal data assistants 1502c, and Network Attached Storage (NAS) 1504a, and 1504b. In example instances, systems 1502a, 1502b, and 1502c can manage data storage and optimize data access for data stored in Network Attached Storage (NAS) 1504a and 1504b. A mathematical model can be used for the data and be evaluated using distributed parallel processing across computer systems 1502a, and 1502b, and cell phone and personal data assistant systems 1502c. Computer systems 1502a, and 1502b, and cell phone and personal data assistant systems 1502c can also provide parallel processing for adaptive data restructuring of the data stored in Network Attached Storage (NAS) 1504a and 1504b. FIG. 15 illustrates an example only, and a wide variety of other computer architectures and systems can be used in conjunction with the various instances of the present disclosure. For example, a blade server can be used to provide parallel processing. Processor blades can be connected through a back plane to provide parallel processing. Storage can also be connected to the back plane or as Network Attached Storage (NAS) through a separate network interface. In some example instances, processors can maintain separate memory spaces and transmit data through network interfaces, back plane or other connectors for parallel processing by other processors. In other instances, some or all of the processors can use a shared virtual address memory space.

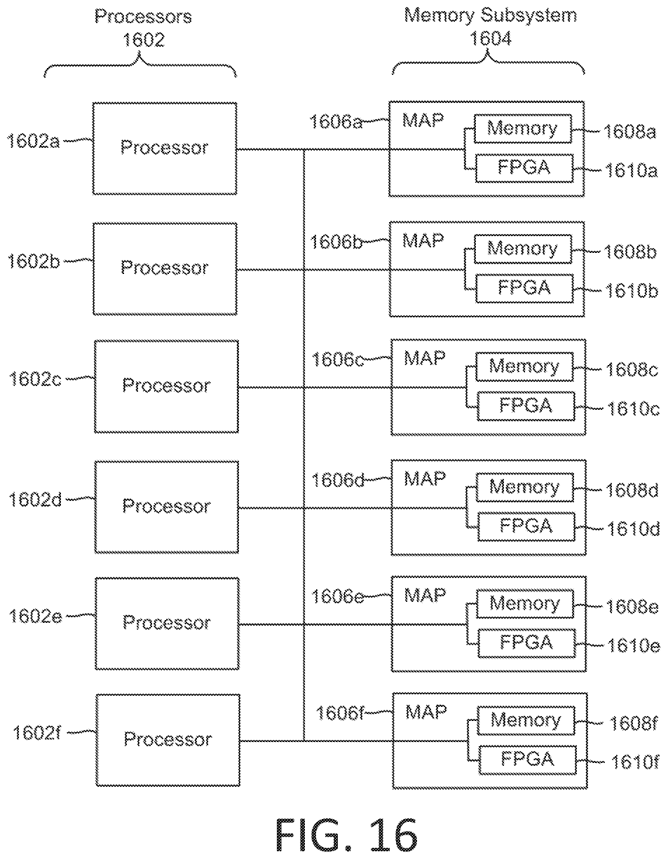

FIG. 16 is a block diagram of a multiprocessor computer system 1600 using a shared virtual address memory space in accordance with an example instance. The system includes a plurality of processors 1602a-f that can access a shared memory subsystem 1604. The system incorporates a plurality of programmable hardware memory algorithm processors (MAPs) 1606s-f in the memory subsystem 1604. Each MAP 1606a-f can comprise a memory 160a-f and one or more field programmable gate arrays (FPGAs) 1610a-f. The MAP provides a configurable functional unit and particular algorithms or portions of algorithms can be provided to the FPGAs 1610a-f for processing in close coordination with a respective processor. For example, the MAPs can be used to evaluate algebraic expressions regarding the data model and to perform adaptive data restructuring in example instances. In this example, each MAP is globally accessible by all of the processors for these purposes. In one configuration, each MAP can use Direct Memory Access (DMA) to access an associated memory 1606a-f, allowing it to execute tasks independently of, and asynchronously from the respective microprocessor 1602a-f. In this configuration, a MAP can feed results directly to another MAP for pipelining and parallel execution of algorithms.

The above computer architectures and systems are examples only, and a wide variety of other computer, cell phone, and personal data assistant architectures and systems can be used in connection with example instances, including systems using any combination of general processors, co-processors, FPGAs and other programmable logic devices, system on chips (SOCs), application specific integrated circuits (ASICs), and other processing and logic elements. In some instances, all or part of the computer system can be implemented in software or hardware. Any variety of data storage media can be used in connection with example instances, including random access memory, hard drives, flash memory, tape drives, disk arrays, Network Attached Storage (NAS) and other local or distributed data storage devices and systems.

In example instances, the computer system can be implemented using software modules executing on any of the above or other computer architectures and systems. In other instances, the functions of the system can be implemented partially or completely in firmware, programmable logic devices such as field programmable gate arrays (FPGAs) as referenced in FIG. 13, system on chips (SOCs), application specific integrated circuits (ASICs), or other processing and logic elements. For example, the Set Processor and Optimizer can be implemented with hardware acceleration through the use of a hardware accelerator card, such as accelerator card 1322 illustrated in FIG. 13.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1: Functionalization of a Device Surface

A device was functionalized to support the attachment and synthesis of a library of oligonucleic acids. The device surface was first wet cleaned using a piranha solution comprising 90% H₂SO₄ and 10% H₂O₂ for 20 minutes. The device was rinsed in several beakers with DI water, held under a DI water gooseneck faucet for 5 min, and dried with N2. The device was subsequently soaked in NH₄OH (1:100; 3 mL:300 mL) for 5 min, rinsed with DI water using a handgun, soaked in three successive beakers with DI water for 1 min each, and then rinsed again with DI water using the handgun. The device was then plasma cleaned by exposing the device surface to O₂. A SAMCO PC-300 instrument was used to plasma etch O₂ at 250 watts for 1 min in downstream mode.

The cleaned device surface was actively functionalized with a solution comprising N-(3-triethoxysilylpropyl)-4-hydroxybutyramide using a YES-1224P vapor deposition oven system with the following parameters: 0.5 to 1 torr, 60 min, 70° C., 135° C. vaporizer. The device surface was resist coated using a Brewer Science 200X spin coater. SPR™ 3612 photoresist was spin coated on the device at 2500 rpm for 40 sec. The device was pre-baked for 30 min at 90° C. on a Brewer hot plate. The device was subjected to photolithography using a Karl Suss MA6 mask aligner instrument. The device was exposed for 2.2 sec and developed for 1 min in MSF 26A. Remaining developer was rinsed with the handgun and the device soaked in water for 5 min. The device was baked for 30 min at 100° C. in the oven, followed by visual inspection for lithography defects using a Nikon 1200. A descum process was used to remove residual resist using the SAMCO PC-300 instrument to O₂ plasma etch at 250 watts for 1 min.

The device surface was passively functionalized with a 100 μL solution of perfluorooctyltrichlorosilane mixed with 10 μL light mineral oil. The device was placed in a chamber, pumped for 10 min, and then the valve was closed to the pump and left to stand for 10 min. The chamber was vented to air. The device was resist stripped by performing two soaks for 5 min in 500 mL NMP at 70° C. with ultrasonication at maximum power (9 on Crest system). The device was then soaked for 5 min in 500 mL isopropanol at room temperature with ultrasonication at maximum power. The device was dipped in 300 mL of 200 proof ethanol and blown dry with N2. The functionalized surface was activated to serve as a support for oligonucleic acid synthesis.

### Example 2: Synthesis of a 50-Mer Sequence on an Oligonucleotide Synthesis Device

A two dimensional oligonucleotide synthesis device was assembled into a flowcell, which was connected to a flowcell (Applied Biosystems (ABI394 DNA Synthesizer”). The two-dimensional oligonucleotide synthesis device was uniformly functionalized with N-(3-TRIETHOXYSILYLPROPYL)-4-HYDROXYBUTYRAMIDE (Gelest) was used to synthesize an exemplary oligonucleotide of 50 bp (“50-mer oligonucleotide”) using oligonucleotide synthesis methods described herein.

The sequence of the 50-mer was as described in SEQ ID NO.: 20. 5′AGACAATCAACCATITGGGGTGGACAGCCTTGACCTCTAGACTTCGGCAT##TTTTT ITITT3′ (SEQ ID NO.: 20), where #denotes Thymidine-succinyl hexamide CED phosphoramidite (CLP-2244 from ChemGenes), which is a cleavable linker enabling the release of oligos from the surface during deprotection.

The synthesis was done using standard DNA synthesis chemistry (coupling, capping, oxidation, and deblocking) according to the protocol in Table 2 and an ABI synthesizer.

TABLE 2

| General DNA Synthesis | Table 2 | |
|---|---|---|
| Process Name | Process Step | Time (sec) |
| WASH (Acetonitrile Wash | Acetonitrile System Flush | 4 |
| Flow) | Acetonitrile to Flowcell | 23 |
| | N2 System Flush | 4 |
| | Acetonitrile System Flush | 4 |
| DNA BASE ADDITION | Activator Manifold Flush | 2 |
| (Phosphoramidite + | Activator to Flowcell | 6 |
| Activator Flow) | Activator + | 6 |
| | Phosphoramidite to | |
| | Flowcell | |
| | Activator to Flowcell | 0.5 |
| | Activator + | 5 |
| | Phosphoramidite to | |
| | Flowcell | |
| | Activator to Flowcell | 0.5 |
| | Activator + | 5 |
| | Phosphoramidite to | |
| | Flowcell | |
| | Activator to Flowcell | 0.5 |
| | Activator + | 5 |
| | Phosphoramidite to | |
| | Flowcell | |
| | Incubate for 25 sec | 25 |
| WASH (Acetonitrile Wash | Acetonitrile System Flush | 4 |
| Flow) | Acetonitrile to Flowcell | 15 |
| | N2 System Flush | 4 |
| | Acetonitrile System Flush | 4 |
| DNA BASE ADDITION | Activator Manifold Flush | 2 |
| (Phosphoramidite + | Activator to Flowcell | 5 |
| Activator Flow) | Activator + | 18 |
| | Phosphoramidite to | |
| | Flowcell | |
| | Incubate for 25 sec | 25 |
| WASH (Acetonitrile Wash | Acetonitrile System Flush | 4 |
| Flow) | Acetonitrile to Flowcell | 15 |
| | N2 System Flush | 4 |
| | Acetonitrile System Flush | 4 |
| CAPPING (CapA + B, 1:1, | CapA + B to Flowcell | 15 |
| Flow) | | |
| WASH (Acetonitrile Wash | Acetonitrile System Flush | 4 |
| Flow) | Acetonitrile to Flowcell | 15 |
| | Acetonitrile System Flush | 4 |
| OXIDATION (Oxidizer | Oxidizer to Flowcell | 18 |
| Flow) | | |
| WASH (Acetonitrile Wash | Acetonitrile System Flush | 4 |
| Flow) | N2 System Flush | 4 |
| | Acetonitrile System Flush | 4 |
| | Acetonitrile to Flowcell | 15 |
| | Acetonitrile System Flush | 4 |
| | Acetonitrile to Flowcell | 15 |
| | N2 System Flush | 4 |
| | Acetonitrile System Flush | 4 |
| | Acetonitrile to Flowcell | 23 |
| | N2 System Flush | 4 |
| | Acetonitrile System Flush | 4 |
| DEBLOCKING (Deblock | Deblock to Flowcell | 36 |
| Flow) | | |
| WASH (Acetonitrile Wash | Acetonitrile System Flush | 4 |
| Flow) | N2 System Flush | 4 |
| | Acetonitrile System Flush | 4 |
| | Acetonitrile to Flowcell | 18 |
| | N2 System Flush | 4.13 |
| | Acetonitrile System Flush | 4.13 |
| | Acetonitrile to Flowcell | 15 |

The phosphoramidite/activator combination was delivered similar to the delivery of bulk reagents through the flowcell. No drying steps were performed as the environment stays “wet” with reagent the entire time.

The flow restrictor was removed from the ABI 394 synthesizer to enable faster flow. Without flow restrictor, flow rates for amidites (0.1M in ACN), Activator, (0.25M Benzoylthiotetrazole (“BTT”; 30-3070-xx from GlenResearch) in ACN), and Ox (0.02M 12 in 20% pyridine, 10% water, and 70% THF) were roughly ˜100 uL/sec, for acetonitrile (“ACN”) and capping reagents (1:1 mix of CapA and CapB, wherein CapA is acetic anhydride in THF/Pyridine and CapB is 16% 1-methylimidizole in THF), roughly ˜200 uL/sec, and for Deblock (3% dichloroacetic acid in toluene), roughly ˜300 uL/sec (compared to ˜50 uL/sec for all reagents with flow restrictor). The time to completely push out Oxidizer was observed, the timing for chemical flow times was adjusted accordingly and an extra ACN wash was introduced between different chemicals. After oligonucleotide synthesis, the chip was deprotected in gaseous ammonia overnight at 75 psi. Five drops of water were applied to the surface to recover oligonucleic acids. The recovered oligonucleic acids were then analyzed on a BioAnalyzer small RNA chip (data not shown).

### Example 3: Synthesis of a 100-Mer Sequence on an Oligonucleotide Synthesis Device

The same process as described in Example 2 for the synthesis of the 50-mer sequence was used for the synthesis of a 100-mer oligonucleotide (“100-mer oligonucleotide”; 5′ CGGGATCCTTATCGTCATCGTCGTACAGATCCCGACCCATTTGCTGTCCACCAGTCAT GCTAGCCATACCATGATGATGATGATGATGAGAACCCCGCAT##TTTTTTTTTT3′, where #denotes Thymidine-succinyl hexamide CED phosphoramidite (CLP-2244 from ChemGenes); SEQ ID NO.: 21) on two different silicon chips, the first one uniformly functionalized with N-(3-TRIETHOXYSILYLPROPYL)-4-HYDROXYBUTYRAMIDE and the second one functionalized with 5/95 mix of 11-acetoxyundecyltriethoxysilane and n-decyltriethoxysilane, and the oligonucleic acids extracted from the surface were analyzed on a BioAnalyzer instrument (data not shown).

All ten samples from the two chips were further PCR amplified using a forward (5′ATGCGGGGTrCTCATCATC3′; SEQ ID NO.: 22) and a reverse (5′CGGGATCCTATCGTCATCG3′; SEQ ID NO.: 23) primer in a 50 uL PCR mix (25 uL NEB Q5 mastermix, 2.5 uL 10 uM Forward primer, 2.5 uL 10 uM Reverse primer, 1 uL oligonucleic acid extracted from the surface, and water up to 50 uL) using the following thermalcycling program:
98 C, 30 sec
98 C, 10 sec; 63 C, 10 sec; 72 C, 10 sec; repeat 12 cycles
72 C, 2 min

The PCR products were also run on a BioAnalyzer (data not shown), demonstrating sharp peaks at the 100-mer position. Next, the PCR amplified samples were cloned, and Sanger sequenced. Table 3 summarizes the results from the Sanger sequencing for samples taken from spots 1-5 from chip 1 and for samples taken from spots 6-10 from chip 2.

TABLE 3

| Spot | Error rate | | Cycle efficiency |
|---|---|---|---|
| 1 | 1/763 | bp | 99.87% |
| 2 | 1/824 | bp | 99.88% |
| 3 | 1/780 | bp | 99.87% |
| 4 | 1/429 | bp | 99.77% |
| 5 | 1/1525 | bp | 99.93% |
| 6 | 1/1615 | bp | 99.94% |
| 7 | 1/531 | bp | 99.81% |
| 8 | 1/1769 | bp | 99.94% |
| 9 | 1/854 | bp | 99.88% |
| 10 | 1/1451 | bp | 99.93% |

Thus, the high quality and uniformity of the synthesized oligonucleotides were repeated on two chips with different surface chemistries. Overall, 89% corresponding to 233 out of 262 of the 100-mers that were sequenced were perfect sequences with no errors.

Finally, Table 4 summarizes error characteristics for the sequences obtained from the oligonucleotides samples from spots 1-10.

TABLE 4

| | Sample ID/Spot no. | | | | |
|---|---|---|---|---|---|
| | OSA_0046/1 | OSA_0047/2 | OSA_0048/3 | OSA_0049/4 | OSA_0050/5 |
| Total Sequences | 32 | 32 | 32 | 32 | 32 |
| Sequencing Quality | 25 of 28 | 27 of 27 | 26 of 30 | 21 of 23 | 25 of 26 |
| Oligo Quality | 23 of 25 | 25 of 27 | 22 of 26 | 18 of 21 | 24 of 25 |
| ROI Match Count | 2500 | 2698 | 2561 | 2122 | 2499 |
| ROI Mutation | 2 | 2 | 1 | 3 | 1 |
| ROI Multi Base | 0 | 0 | 0 | 0 | 0 |
| Deletion | | | | | |
| ROI Small Insertion | 1 | 0 | 0 | 0 | 0 |
| ROI Single Base | 0 | 0 | 0 | 0 | 0 |
| Deletion | | | | | |
| Large Deletion Count | 0 | 0 | 1 | 0 | 0 |
| Mutation: G > A | 2 | 2 | 1 | 2 | 1 |
| Mutation: T > C | 0 | 0 | 0 | 1 | 0 |
| ROI Error Count | 3 | 2 | 2 | 3 | 1 |
| ROI Error Rate | Err: ~1 | Err: ~1 | Err: ~1 | Err: ~1 | Err: ~1 |
| | in 834 | in 1350 | in 1282 | in 708 | in 2500 |
| ROI Minus Primer | MP | MP | MP | MP | MP |
| Error Rate | Err: ~1 | Err: ~1 | Err: ~1 | Err: ~1 | Err: ~1 |
| | in 763 | in 824 | in 780 | in 429 | in 1525 |
| | Sample ID/Spot no. | | | | |
| | OSA_0051/6 | OSA_0052/7 | OSA_0053/8 | OSA_0054/9 | OSA_0055/10 |
| Total Sequences | 32 | 32 | 32 | 32 | 32 |
| Sequencing Quality | 29 of 30 | 27 of 31 | 29 of 31 | 28 of 29 | 25 of 28 |
| Oligo Quality | 25 of 29 | 22 of 27 | 28 of 29 | 26 of 28 | 20 of 25 |
| ROI Match Count | 2666 | 2625 | 2899 | 2798 | 2348 |
| ROI Mutation | 0 | 2 | 1 | 2 | 1 |
| ROI Multi Base | 0 | 0 | 0 | 0 | 0 |
| Deletion | | | | | |
| ROI Small Insertion | 0 | 0 | 0 | 0 | 0 |
| ROI Single Base | 0 | 0 | 0 | 0 | 0 |
| Deletion | | | | | |
| Large Deletion Count | 1 | 1 | 0 | 0 | 0 |
| Mutation: G > A | 0 | 2 | 1 | 2 | 1 |
| Mutation: T > C | 0 | 0 | 0 | 0 | 0 |
| ROI Error Count | 1 | 3 | 1 | 2 | 1 |
| ROI Error Rate | Err: ~1 | Err: ~1 | Err: ~1 | Err: ~1 | Err: ~1 |
| | in 2667 | in 876 | in 2900 | in 1400 | in 2349 |
| ROI Minus Primer | MP | MP | MP | MP | MP |
| Error Rate | Err: ~1 | Err: ~1 | Err: ~1 | Err: ~1 | Err: ~1 |
| | in 1615 | in 531 | 1769 | in 854 | 1451 |

### Example 4: Generation of an Oligonucleic Add Library by Single-Site, Mingle Position Mutagenesis

Oligonucleic acid primers were de novo synthesized for use in a series of PCR reactions to generate a library of oligonucleic acid variants of a template nucleic acid, see FIGS. 2A-21D. Four types of primers were generated in FIG. 2A: an outer 5′ primer 215, an outer 3′ primer 230, an inner 5′ primer 225, and an inner 3′ primer 220. The inner 5′ primer/first oligonucleic acid 220 and an inner 3′ primer/second oligonucleic acid 225 were generated using an oligonucleic acid synthesis method as generally outlined in Table 2. The inner 5′ primer/first oligonucleic acid 220 represents a set of up to 19 primers of predetermined sequence, where each primer in the set differs from another at a single codon, in a single site of the sequence.

Oligonucleic acid synthesis was performed on a device having at least two clusters, each cluster having 121 individually addressable loci.

The inner 5′ primer 225 and the inner 3′ primer 220 were synthesized in separate clusters. The inner 5′ primer 225 was replicated 121 times, extending on 121 loci within a single cluster. For inner 3′ primer 220, each of the 19 primers of variant sequences were each extended on 6 different loci, resulting in the extension of 114 oligonucleic acids on 114 different loci.

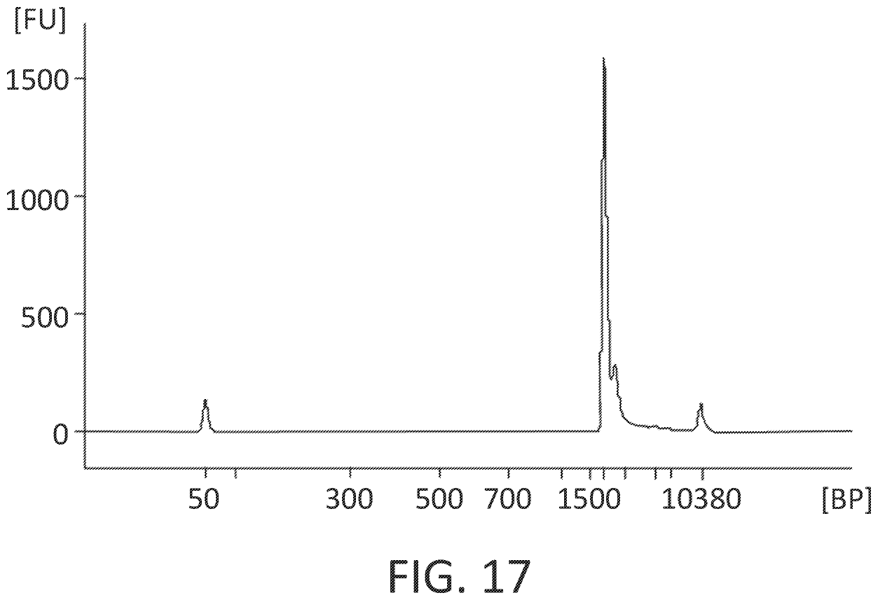

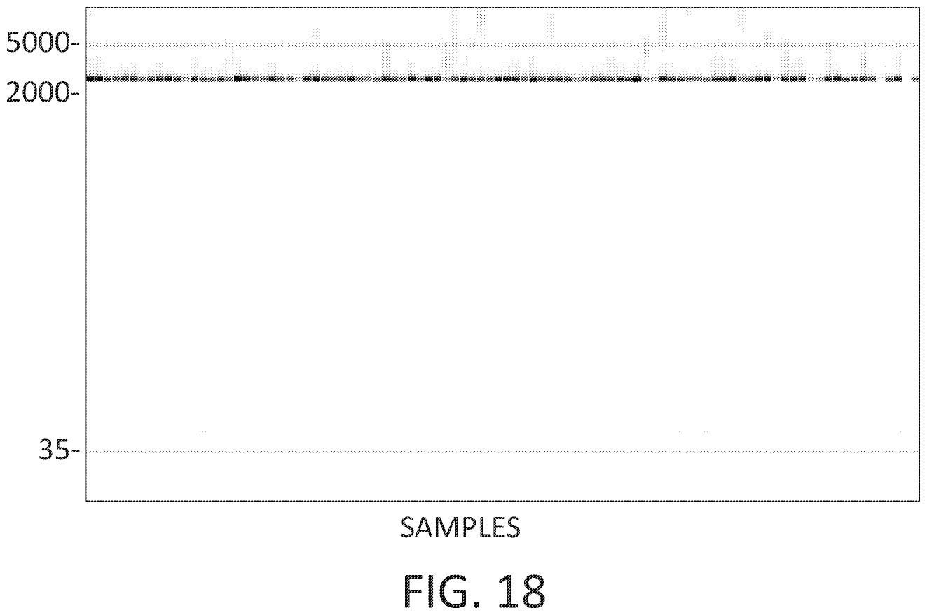

Synthesized oligonucleic acids were cleaved from the surface of the device and transferred to a plastic vial. A first PCR reaction was performed, using fragments of the long nucleic acid sequence 235, 240 to amplify the template nucleic acid, as illustrated in FIG. 23. A second PCR reaction was performed using primer combination and the products of the first PCR reaction as a template, as illustrated in FIGS. 2C-2D. Analysis of the second PCR products was conducted on a BioAnalyzer, as shown in the trace of FIG. 17.

### Example 5: Generation of an Oligonucleic Aid Library Comprising 96 Different Sets of Single Position Variants

Four sets of primers, as generally shown in FIG. 2A and addressed in Example 2, were generated using de novo oligonucleic acid synthesis. For the inner 5′ primer 220, % different sets of primers were generated, each set of primers targeting a different single codon positioned within a single site of the template oligonucleic acid. For each set of primers, 19 different variants were generated, each variant comprising a codon encoding for a different amino acid at the single site. Two rounds of PCR were performed using the generated primers, as generally shown in FIGS. 2A-2D and described in Example 2. The 96 sets of amplification products were visualized in an electropherogram (FIG. 18), which was used to calculate a 100% amplification success rate.

### Example 6: Generation of an Oligonucleic Acid Library Comprising 500 Different Sets of Single Position Variants

Four sets of primers, as generally shown in FIG. 2A and addressed in Example 2, were generated using de novo oligonucleic acid synthesis. For the inner 5′ primer 220, 500 different sets of primers were generated, each set of primers targeting a different single codon positioned within a single site of the template oligonucleic acid. For each set of primers, 19 different variants were generated, each variant comprising a codon encoding for a different amino acid at the single site. Two rounds of PCR were performed using the generated primers, as generally shown in FIG. 2A and described in Example 2. Electropherograms display each of the 500 sets of PCR products having a population of nucleic acids with 19 variants at a different single site (data not shown). A comprehensive sequencing analysis of the library showed a greater than 99% success rate across preselected codon mutations (sequence trace and analysis data not shown).

### Example 7: Single-Site Mutagenesis Primers for 1 Position

An example of codon variation design is provided in Table 3 for Yellow Fluorescent Protein. In this case, a single codon from a 50-mer of the sequence is varied 19 times. Variant nucleic acid sequence is indicated by bold letters. The wild type primer sequence is:

(SEQ ID NO.: 1)
ATGGTGAGCAAGGGCGAGGAGCTGTTCACCGGGGTGGTGCCCAT.

In this case, the wild type codon encodes for valine, indicated by underline in SEQ ID NO.: 1. Therefore the 19 variants below excludes a codon encoding for valine. In an alternative example, if all triplets are to be considered, then all 60 variants would be generated, including alternative sequence for the wild type codon.

TABLE 3

| SEQ | | |
|---|---|---|
| ID | | Variant |
| NO. | Variant sequence | codon |
| 2 | atgTTTAGCAAGGGCGAGGAGC | F |
| | TGTTCACCGGGGTGGTGCCCAT | |
| 3 | atgTTAAGCAAGGGCGAGGAGC | L |
| | TGTTCACCGGGGTGGTGCCCAT | |
| 4 | atgATTAGCAAGGGCGAGGAGC | I |
| | TGTTCACCGGGGTGGTGCCCAT | |
| 5 | atgTCTAGCAAGGGCGAGGAGC | S |
| | TGTTCACCGGGGTGGTGCCCAT | |
| 6 | atgCCTAGCAAGGGCGAGGAGC | P |
| | TGTTCACCGGGGTGGTGCCCAT | |
| 7 | atgACTAGCAAGGGCGAGGAGC | T |
| | TGTTCACCGGGGTGGTGCCCAT | |
| 8 | atgGCTAGCAAGGGCGAGGAGC | A |
| | TGTTCACCGGGGTGGTGCCCAT | |
| 9 | atgTATAGCAAGGGCGAGGAGC | Y |
| | TGTTCACCGGGGTGGTGCCCAT | |
| 10 | atgCATAGCAAGGGCGAGGAGC | H |
| | TGTTCACCGGGGTGGTGCCCAT | |
| 11 | atgCAAAGCAAGGGCGAGGAGC | Q |
| | TGTTCACCGGGGTGGTGCCCAT | |
| 12 | atgAATAGCAAGGGCGAGGAGC | N |
| | TGTTCACCGGGGTGGTGCCCAT | |
| 13 | atgAAAAGCAAGGGCGAGGAGC | K |
| | TGTTCACCGGGGTGGTGCCCAT | |
| 14 | atgGATAGCAAGGGCGAGGAGC | D |
| | TGTTCACCGGGGTGGTGCCCAT | |
| 15 | atgGAAAGCAAGGGCGAGGAGC | E |
| | TGTTCACCGGGGTGGTGCCCAT | |
| 16 | atgTGTAGCAAGGGCGAGGAGC | C |
| | TGTTCACCGGGGTGGTGCCCAT | |
| 17 | atgTGGAGCAAGGGCGAGGAGC | W |
| | TGTTCACCGGGGTGGTGCCCAT | |
| 18 | atgCGTAGCAAGGGCGAGGAGC | R |
| | TGTTCACCGGGGTGGTGCCCAT | |
| 19 | atgGGTAGCAAGGGCGAGGAGC | G |
| | TGTTCACCGGGGTGGTGCCCAT | |

### Example 8: Single Site, Dual Position Oligonucleic Acid Variants

De novo oligonucleic acid synthesis was performed under conditions similar to those described in Example 2. A single cluster on a device was generated which contained synthesized predetermined variants of an oligonucleic acid for 2 consecutive codon positions at a single site, each position being a codon encoding for an amino acid. In this arrangement, 19 variants/per position were generated for 2 positions with 3 replicates of each oligonucleic acid, resulting in 114 oligonucleic acids synthesized.

### Example 9: Multiple Site, Dual Position Oligonucleic Acid Variants

De novo oligonucleic acid synthesis was performed under conditions similar to those described in Example 2. A single cluster on a device was generated which contained synthesized predetermined variants of an oligonucleic acid for 2 non-consecutive codon positions, each position being a codon encoding for an amino acid. In this arrangement, 19 variants/per position were generated for 2 positions.

### Example 10: Single Stretch, Triple Position Oligonucleic Acid Variants

De novo oligonucleic acid synthesis was performed under conditions similar to those described in Example 2. A single cluster on a device was generated which contained synthesized predetermined variants of a reference oligonucleic acid for 3 consecutive codon positions. In the 3 consecutive codon position arrangement, 19 variants/per position were generated for 3 positions with 2 replicates of each oligonucleic acid, and resulted in 114 oligonucleic acids synthesized.

### Example 11: Multiple Site, Triple Position Oligonucleic Acid Variants

De novo oligonucleic acid synthesis was performed under conditions similar to those described in Example 2. A single cluster on a device was generated which contains synthesized predetermined variants of a reference oligonucleic acid for at least 3 non-consecutive codon positions. Within a predetermined region, the location of codons encoding for 3 histidine residues were varied.

### Example 12: Multiple Site, Multiple Position Oligonucleic Acid Variants

De novo oligonucleic acid synthesis was performed under conditions similar to those described in Example 2. A single cluster on a device was generated which contained synthesized predetermined variants of a reference oligonucleic acid for 1 or more codon positions in 1 or more stretches. Five positions were varied in the library. The first position encoded codons for a resultant 50/50 K/R ratio in the expressed protein; the second position encoded codons for a resultant 50/25/25 V/L/S ratio in the expressed protein, the third position encoded codons for a resultant a 50/25/25 Y/R/D ratio in the expressed protein, the fourth position encoded codons for a resultant an equal ratio for all amino acids in the expressed protein, and the fifth position encoded codons for a resultant a 75/25 G/P ratio in the expressed protein.

### Example 13: Stretch in a CDR Having Multiple Variant Sites

An oligonucleotide library is generated as in Examples 4-6 and 8-12, encoding for codon variation at a single site or multiple sites where variants are preselected at each position. The variant region encodes for at least a portion of a CDR. See, for example, FIG. 10. Synthesized oligonucleic acids are released from the device surface, and used as primers to generate a nucleic acid library, which is expressed in cells to generate a variant protein library. Variant antibodies are assessed for increase binding affinity to an epitope.

### Example 14: Modular Plasmid Components for Expressing Diverse Peptides

An oligonucleotide library is generated as in Examples 4-6 and 8-12, encoding for codon variation at a single site or multiple sites for each of separate regions that make up portions of an expression construct cassette, as depicted in FIG. 11. To generate a two construct expressing cassette, variant oligonucleic acids were synthesized encoding at least a portion of a variant sequence of a first promoter 1110, first open reading frame 1120, first terminator 1130, second promoter 1140, second open reading frame 1150, or second terminator sequence 1160. After rounds of amplification, as described in previous examples, a library of 1,024 expression constructs is generated.

### Example 15: Multiple Site, Single Position Variants

An oligonucleotide library is generated as in Examples 4-6 and 8-12, encoding for codon variation at a single site or multiple sites in a region encoding for at least a portion of nucleic acid. A library of oligonucleic acid variants is generated, wherein the library consists of multiple site, single position variants. See, for example, FIG. 6B.

### Example 16: Variant Library Synthesis

De novo oligonucleic acid synthesis is performs under conditions similar to those described in Example 2. At least 30,000 non-identical oligonucleic acids are de novo synthesized, wherein each of the non-identical oligonucleic acids encodes for a different codon variant of an amino acid sequence. The synthesized at least 30,000 non-identical oligonucleic acids have an aggregate error rate of less than 1 in 1:000 bases compared to predetermined sequences for each of the at least 30,000 non-identical oligonucleic acids. The library is used for PCR mutagenesis of a long nucleic acid and at least 30,000 non-identical variant nucleic acids are formed.

### Example 17: Variant Library Synthesis in a Well

De novo oligonucleic acid synthesis is performs under conditions similar to those described in Example 2. A single cluster on a device is generated which contained synthesized predetermined variants of a reference oligonucleic acid for 2 codon positions. In the 2 consecutive codon position arrangement, 19 variants/per position were generated for the 2 positions with 2 replicates of each oligonucleic acid, and resulted in 38 oligonucleic acids synthesized. Each variant sequence is 40 bases in length. In the same cluster, additional non-variant oligonucleic acids sequence are generated, where the additional non-variant oligonucleic acids and the variant nucleic acids collective encode for 38 variants of the coding sequence of a gene. Each of the oligonucleic acids has at least one region reverse complementary to another of the oligonucleic acids. The oligonucleic acids in the cluster are released by gaseous ammonia cleavage. A pin comprising water contacts the cluster, picks up the oligonucleic acids, and moves the oligonucleic acids to a small vial. The vial also contains DNA polymerase reagents for a polymerase cycling assembly (PCA) reaction. The oligonucleic acids anneal, gaps are filled in by an extension reaction, and resultant double-stranded DNA molecules are formed, forming a variant nucleic acid library. The variant nucleic acid library is, optionally, subjected to restriction enzyme is then ligated into expression vectors.

### Example 19: Screening a Variant Nucleic Acid Library for Changes in Protein Binding Affinity

A plurality of expression vectors are generated as described in Examples 16 or 17. In this example, the expression vector is a HIS-tagged bacterial expression vector. The vector library is electroporated into bacterial cells and then clones are selected for expression and purification of HIS-tagged variant proteins. The variant proteins are screened for a change binding affinity to a target molecule.

Affinity is examined by methods such as using metal affinity chromatography (IMAC), where a metal ion coated resin (e.g., IDA-agarose or NTA-agarose) is used to isolate HIS-tagged proteins. Expressed His-tagged proteins can be purified and detected because the string of histidine residues binds to several types of immobilized metal ions, including nickel, cobalt and copper, under specific buffer conditions. An example binding/wash buffer consists of Tris-buffer saline (TBS) pH 7.2, containing 10-25 mM imidazole. Elution and recovery of captured His-tagged protein from an IMAC column is accomplished with a high concentration of imidazole (at least 200 mM) (the elution agent), low pH (e.g., 0.1M glycine-HCl, pH 2.5) or an excess of strong chelator (e.g., EDTA).

Alternatively, anti-His-tag antibodies are commercially available for use in assay methods involving His-tagged proteins, such as a pull-down assay to isolate His-tagged proteins or an immunoblotting assay to detect His-tagged proteins.

### Example 19: Screening a Variant Nucleic Acid Library for Changes in Activity for a Regulator of Cell Adhesion and Migration

A plurality of expression vectors are generated as described in Examples 16 or 17. In this example, the expression vector is a GFP-tagged mammalian expression vector. Isolated clones from the library are transiently transfected into mammalian cells. Alternatively, proteins are expressed and isolated from cells containing the expression constructs, and then the proteins are delivered to cells for further measurements. Immunofluorescent assays are conducted to assess changes in cellular localization of the GFP-tagged variant expression products. FACS assays are conduct to assess changes in the conformational state of a transmembrane protein that interacts with a non-variant version of a GFP-tagged variant protein expression product. Wound healing assays are conducted to assess changes in the ability of cells expressing a GFP-tagged variant protein to invade space created by a scrape on a cell culture dish. Cells expressing GFP-tagged proteins are identified and tracked using a fluorescent light source and a camera.

### Example 20: Screening a Variant Nucleic Acid Library for Peptides Inhibiting Viral Progression

A plurality of expression vectors are generated as described in Examples 16 or 17. In this example, the expression vector is a FLAG-tagged mammalian expression vector and the variant nucleic acid library encodes for peptide sequences. Primary mammalian cells are obtained from a subject suffering from a viral disorder. Alternatively, primary cells from a healthy subject are infected with a virus. Cells are plated on a series of microwell dishes. Isolated clones from the variant library are transiently transfected into the cells. Alternatively, proteins are expressed and isolated from cells containing the expression constructs, and then the proteins are delivered to cells for further measurements. Cell survival assays are performed to assess infected cells for enhanced survival associated with a variant peptide. Exemplary viruses include, without limitation, avian flu, zika virus, Hantavirus, Hepatitis C, smallpox,

One example assay is the neutral red cytotoxicity assay which uses neutral red dye, that, when added to cells, diffuses across the plasma membrane and accumulates in the acidic lysosomal compartment due to the mildly cationic properties of neutral red. Virus-induced cell degeneration leads to membrane fragmentation and loss of lysosome ATP-driven proton translocating activity. The consequent reduction of intracellular neutral red can be assessed spectrophotometrically in a multi-well plate format. Cells expressing variant peptides are scored by an increase in intracellular neutral red in a gain-of-signal color assay. Cells are assessed for peptides inhibiting virus-induced cell degeneration.

### Example 21: Screening for Variant Proteins that Increase or Decrease Metabolic Activity of a Cell

A plurality of expression vectors are generated as described in Examples 16 or 17 for the purpose of identifying expression products that result in a change in metabolic activity of a cell. In this example, the expression vectors are transferred (e.g., via transfection or transduction) into cells plated on a series of microwell dishes. Cells are then screened for one or more changes in metabolic activity. Alternatively, proteins are expressed and isolated from cells containing the expression constructs, and then the proteins are delivered to cells for measuring metabolic activity. Optionally, cells for measuring metabolic activity are treated with a toxin prior to screening for one or more changes metabolic activity. Exemplary toxins administered included, without limitation, botulinum toxin (including immunological types: A, B, C1, C2, D, E, F, and G), staphylococcus enterotoxin B, Yersinia pestis, Hepatitis C, Mustard agents, heavy metals, cyanide, endotoxin, Bacillus anthracis, zika virus, avian flu, herbicides, pesticides, mercury, organophosphates, and ricin.

The basal energy requirements are derived from the oxidation of metabolic substrates, e.g., glucose, either by oxidative phosphorylation involving the aerobic tricarboxylic acid (TCA) or Kreb's cycle or anaerobic glycolysis. When glycolysis is the major source of energy, the metabolic activity of cells can be estimated by monitoring the rate at which the cells excrete acidic products of metabolism, e.g., lactate and CO₂. In the case of aerobic metabolism, the consumption of extracellular oxygen and the production of oxidative free radicals are reflective of the energy requirements of the cell. Intracellular oxidation-reduction potential can be measured by autofluorescent measurement of the NADH and NAD⁺. The amount of energy, e.g., heat, released by the cell is derived from analytical values for substances produced and/or consumed during metabolism which under normal settings can be predicted from the amount of oxygen consumed (e.g., 4.8 kcal/l O₂). The coupling between heat production and oxygen utilization can be disturbed by toxins. Direct microcalorimetry measures the temperature rise of a thermally isolated sample. Thus when combined with measurements of oxygen consumption calorimetry can be used to detect the uncoupling activity of toxins.

Various methods and devices are known in the art for measuring changes in various marker of metabolic activity. For example, such methods, devices, and markers are discussed in U.S. Pat. No. 7,704,745, which is herein incorporated by reference in its entirety. Briefly, measurement of the any of the following characteristics is recorded for each cell population: glucose, lactate, CO₂, NADH and NAD⁺ ratio, heat, O₂ consumption, and free-radical production. Cells screened can include hepatocyte, macrophages or neuroblastoma cells. Cells screened can cell lines, primary cells from a subject, or cells from a model system (e.g., a mouse model).

Various techniques are available for measurement of the oxygen consumption rates of single cells, or a population of cells located within a chamber of a multiwell plate. For example, chambers comprising the cells can have sensors for recording changes in temperature, current or fluorescence, as well as optical systems, e.g., a fiber-coupled optical system, coupled to each chamber to monitor fluorescent light. In this example, each chamber has a window for an illumination source to excite molecules inside the chamber. The fiber-coupled optical system can detect autofluorescence to measure intracellular NADH/NAD ratios and voltage and calcium-sensitive dyes to determine transmembrane potential and intracellular calcium. In addition, changes in CO₂ and/or O₂ sensitive fluorescent dye signal is detected.

### Example 22: Screening a Variant Nucleic Add Library for Selective Targeting of Cancer Cells

A plurality of expression vectors are generated as described in Examples 16 or 17. In this example, the expression vector is a FLAG-tagged mammalian expression vector and the variant nucleic acid library encodes for peptide sequences. Isolated clones from the variant library are transiently transfected separately into a cancer cells and non-cancer cells. Cell survival and cell death assays are performed on both the cancer and non-cancer cells, each expressing a variant peptide encoded by the variant nucleic. Cells are assessed for selective cancer cell killing associated with a variant peptide. The cancer cells are, optionally, a cancer cell line or primary cancer cells from a subject diagnosed with cancer. In the case of primary cancer cells from a subject diagnosed with cancer, a variant peptide identified in the screening assay is, optionally, selected for administration to the subject. Alternatively, proteins are expressed and isolated from cells containing the protein expression constructs, and then the proteins are delivered to cancer cells and non-cancer cells for further measurements.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1-35. (canceled)

36. A method for generating a nucleic acid library, comprising:
synthesizing a plurality of oligonucleotides encoding a plurality of nucleic acid sequences comprising predetermined ratios of codon sequence variance for multiple codon positions compared to a reference sequence that when expressed comprise variant ratios of amino acids in a protein.

37. The method of claim 36, wherein the plurality of nucleic acid sequences comprise predetermined ratios of codon sequence variance for at least 5 codon positions.

38. The method of claim 36, wherein the plurality of nucleic acid sequences comprise predetermined ratios of codon sequence variance for at least two adjacent codon positions compared to the reference sequence.

39. The method of claim 36, wherein the plurality of nucleic acid sequences comprise predetermined ratios of codon sequence variance for at least two non-adjacent codon positions compared to the reference sequence.

40. The method of claim 36, wherein the plurality of nucleic acid sequences comprise at least 500 nucleic acid sequences.

41. The method of claim 36, wherein each of the plurality of nucleic acid sequences comprises at least 20 bases in length.

42. A method for generating a nucleic acid library, comprising:
a) synthesizing a first plurality of oligonucleotides, wherein the first plurality of oligonucleotides encode for a first plurality of nucleic acid sequences encoding for 19 variants for each position of multiple codon positions compared to a reference sequence;
b) synthesizing a second plurality of oligonucleotides, wherein the second plurality of oligonucleotides encode for a second plurality of nucleic acid sequences encoding for 19 variants for each position of multiple codon positions compared to the reference sequence; and
c) mixing the first plurality of oligonucleotides and the second plurality oligonucleotides without performing an amplification step to form the nucleic acid library.

43. The method of claim 42, wherein the first plurality of nucleic acid sequences or the second plurality of nucleic acid sequences comprise predetermined ratios of codon sequence variance for at least 5 codon positions.

44. The method of claim 42, wherein the first plurality of nucleic acid sequences or the second plurality of nucleic acid sequences comprise predetermined ratios of codon sequence variance for at least two adjacent codon positions compared to the reference sequence.

45. The method of claim 42, wherein the first plurality of nucleic acid sequences or the second plurality of nucleic acid sequences comprise predetermined ratios of codon sequence variance for at least two non-adjacent codon positions compared to the reference sequence.

46. The method of claim 42, wherein the first plurality of nucleic acid sequences or the second plurality of nucleic acid sequences comprise at least 500 nucleic acid sequences.

47. The method of claim 42, wherein each of the first plurality of nucleic acid sequences or each of the second plurality of nucleic acid sequences comprises at least 20 bases in length.

48. A method for generating a nucleic acid library, comprising:
a) synthesizing a first plurality of nucleic acids encoding at least about 10³ variant proteins in a first region;
b) synthesizing a second plurality of nucleic acids encoding at least about I0³ variant proteins in a second region;
c) synthesizing a third plurality of nucleic acids encoding at least about I0³ variant proteins in a third region; and
d) mixing the first plurality of nucleic acids, the second plurality of nucleic acids, and the third plurality of nucleic acids to form a library of variant nucleic acids that when expressed comprise at least about 10⁹ variant proteins.
